(19) United States
(12) Patent Application Publication
(10) Pub. No.: US 2021/0287783 A1
(43) Pub. Date: Sep. 16, 2021

(54) METHODS AND SYSTEMS FOR A WORKFLOW TRACKER

(71) Applicant: GE Precision Healthcare LLC, Milwaukee, WI (US)

(72) Inventor: Niketa Jhaveri, Naperville, IL (US)

(21) Appl. No.: 16/816,146

(22) Filed: Mar. 11, 2020

(51) Int. Cl. G16H 40/20; G16H 15/00; G16H 30/20; G06Q 10/06; G16H 70/20

(52) U.S. Cl. CPC G16H 40/20; G16H 15/00; G16H 70/20; G06Q 10/063114; G06Q 10/06316; G16H 30/20

(57) ABSTRACT

Methods and systems are provided for tracking workflow in healthcare. In one example, a workflow tracker system may monitor and track workflow of a patient, and a workflow status for the patient may be indicated via a workflow tracker interface. In another example, the workflow tracker system may track workflow of one or more care providers involved in the care of a patient.

### FIELD

Embodiments of the subject matter disclosed herein relate to methods for tracking the workflow of one or more of care providers and patients in a medical facility in real-time.

### BACKGROUND

Care of a patient in a hospital or other medical facility may be carried out by multiple care providers and involve the patient undergoing multiple exams and/or types of exams (e.g., an MRI, x-ray imaging). The workflow of the care provider with regard to the patient may depend on multiple factors including the patient's medical history, coordination of patient care among all the care providers, and images/reports/findings generated during the workflow. Thus, to ensure operational efficiency, tracking a care provider's workflow in real-time may be necessary. Further, coordination of patient care among all the care providers may be complicated or time-consuming, further stretching care provider resources. Additionally, the presentation of patient medical information to the care providers may require multiple time-consuming and cumbersome requests or searches for information.

### BRIEF DESCRIPTION

In one embodiment, a method comprises receiving an indication that a patient has been admitted for an exam, and in response, initiating a workflow tracker for the patient; updating the workflow tracker in real-time as the patient progresses through the exam; and outputting a graphical user interface (GUI) for display on a display device, the GUI including an indication of current exam progress as determined by the workflow tracker.

In this way, the workflow tracker enables a user to understand a current status of patient workflow at a glance at the interface. As a result, workflow efficiency increases as time taken to understand the various patient-related information is reduced, which in turn improves patient care.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 schematically shows an example of a workflow tracker system, according to an embodiment of the present disclosure;

FIG. 2 shows a block diagram of an example workflow tracker infrastructure including one or more systems, according to an embodiment of the present disclosure;

FIG. 3A shows an example display device displaying a workflow tracker interface of the workflow tracker, according to an embodiment of the present disclosure;

FIG. 3B shows another exemplary workflow tracker interface, according to an embodiment of the present disclosure;

FIG. 4 is a high-level flow chart for adding information to a workflow tracker system based on a current workflow, according to an embodiment of the present disclosure;

FIG. 5 is a high-level flow chart for tracking a current workflow of a user and providing recommendations based on user status and expected workflow, according to an embodiment of the present disclosure;

FIG. 6 is a high-level flow chart for tracking a current workflow of a user and providing recommendations for a current patient workflow based one or more past patient workflows, according to an embodiment of the present disclosure;

FIG. 7 is an example of a timeline that may be generated by the method of FIG. 6;

FIG. 8 is a high-level flow chart for tracking the workflow of a specific patient case in real-time, according an embodiment of the present disclosure;

FIG. 9 shows example timelines for a first user and a second user involved in a current patient workflow tracked by a workflow tracker system, according to an embodiment of the present disclosure;

FIG. 10 is a flow chart of a method for facilitating communication amongst and outputting notifications to collaborative users based on the tracked workflow, according to an embodiment of the present disclosure;

FIG. 11 shows an example viewer interface displaying the workflow tracker interface of FIG. 3A including display of image information within an image viewer;

FIG. 12 shows an example viewer interface displaying the workflow tracker interface of FIG. 3A including an enlarged portion of the image information shown in FIG. 11; and

FIG. 13 shows an example viewer interface displaying the workflow tracker interface of FIG. 3A including a generated worklist.

### DETAILED DESCRIPTION

Hospitals and medical institutions generally experience financial and operational stress, having thin margins where physical capacity is at a premium. Thus, the health care industry is often faced with pressure to design, or redesign, its workflows to be more efficient and effective. In many cases, workflows may be updated or changed in response to the introduction of new technologies and treatment methodologies, cost and efficiency pressure, the challenges of coordinated care, to improve patient flow/safety, and/or other healthcare initiatives.

Recent changes in medical institution workflows have largely been focused on increasing staff communications and the workflow efficiency related to specific aspects to patient care. For example, efforts have been made to improve the workflow processes related to patient admission and discharge as well diagnostic examinations. However, workflow improvements related to the patient flow from the time of admission to the time of discharge are still lacking. From the time of admission, patients are in a near constant state of motion, as they are moved from waiting rooms to exam rooms, undergo treatment, and so on, leaving a lot of room for unnecessary delays or error. Currently, tracking of a patient case does not exist on a real-time basis. Thus, as the patient moves from one healthcare provider to the next, the current care provider may struggle to understand the case pattern and where case information came from. Tracking down the sources and details behind case information often becomes a time-consuming process for healthcare providers who, due to a multitude of patients, can only devote a small percentage of time to each patient. Thus, patient care may be rushed or rescheduled out of necessity, wait times may be increased, and operational efficiency lowered.

Thus, according to the embodiments disclosed herein, a workflow tracker system may be employed to track the workflow of one or more of healthcare and collaborative care providers with respect to a patient in real-time. The workflow tracker system facilitates communication amongst and information dissemination to one or more of healthcare and collaborative care providers, while tracking and updating a patient worklist with detailed information (e.g., linked images and reports, details of who performed what action and when, etc.) in real-time based on the tracked actions of current users. The workflow tracker system is communicatively coupled to a healthcare information infrastructure and includes a workflow tracker interface so that the patient's previous medical records, current medical information, and worklist may be easily accessed as the case is updated in real-time. Further, the workflow tracker system includes a predictive learning model that may output next steps for the current user based on the real-time tracked workflow of the patient.

By tracking care provider workflows in real-time, institutions may be able to recognize and address efficiency, time, and/or cost related bottlenecks within operations. Appropriate solutions can then be tailored and monitored for common patient problems (e.g., long waiting times, overcrowding, delayed or rescheduled exams, etc.). For example, patient throughput may be increased which may increase revenue, lower costs, increase asset utilization, and improve the patient experience. Further, healthcare providers and/or other users of the workflow tracker system may understand where a patient case came from, where it stands in the current time at a glance, and what potential next steps to take, thereby saving time and potential stress.

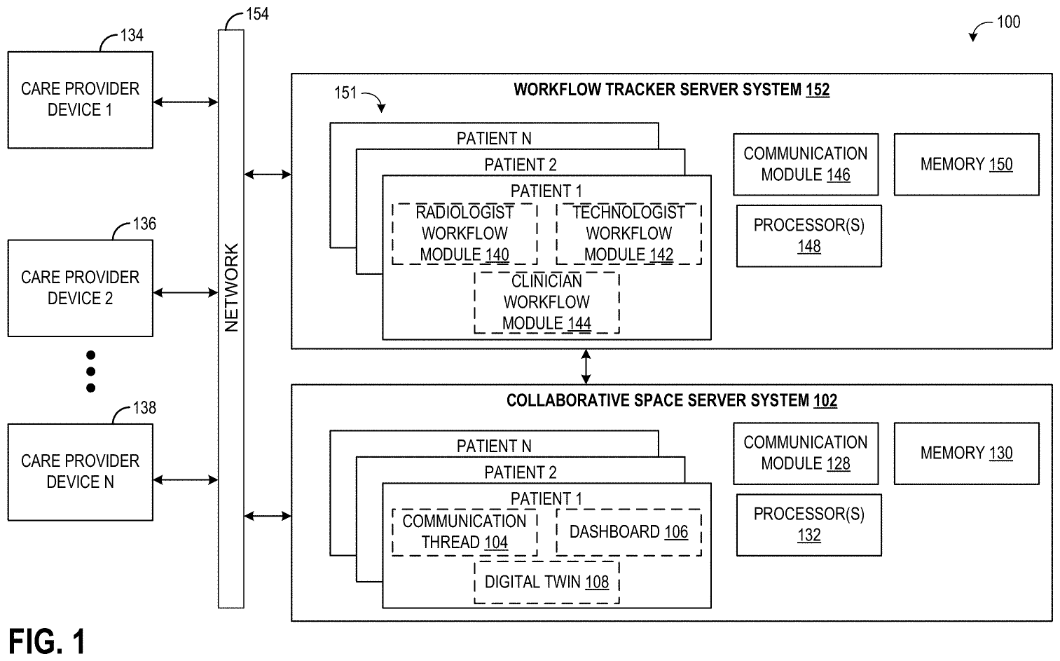

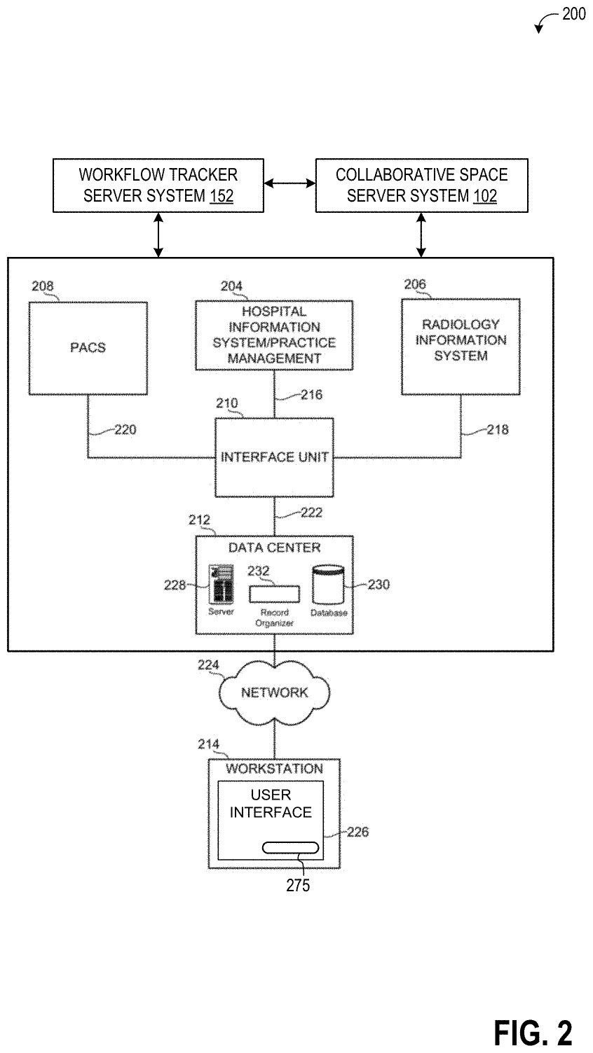

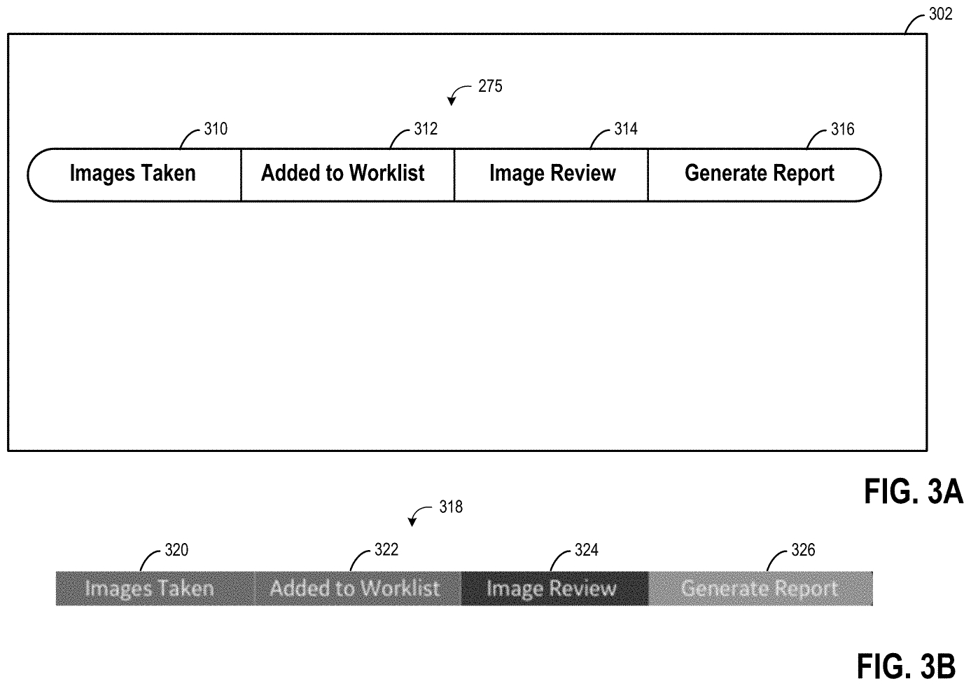

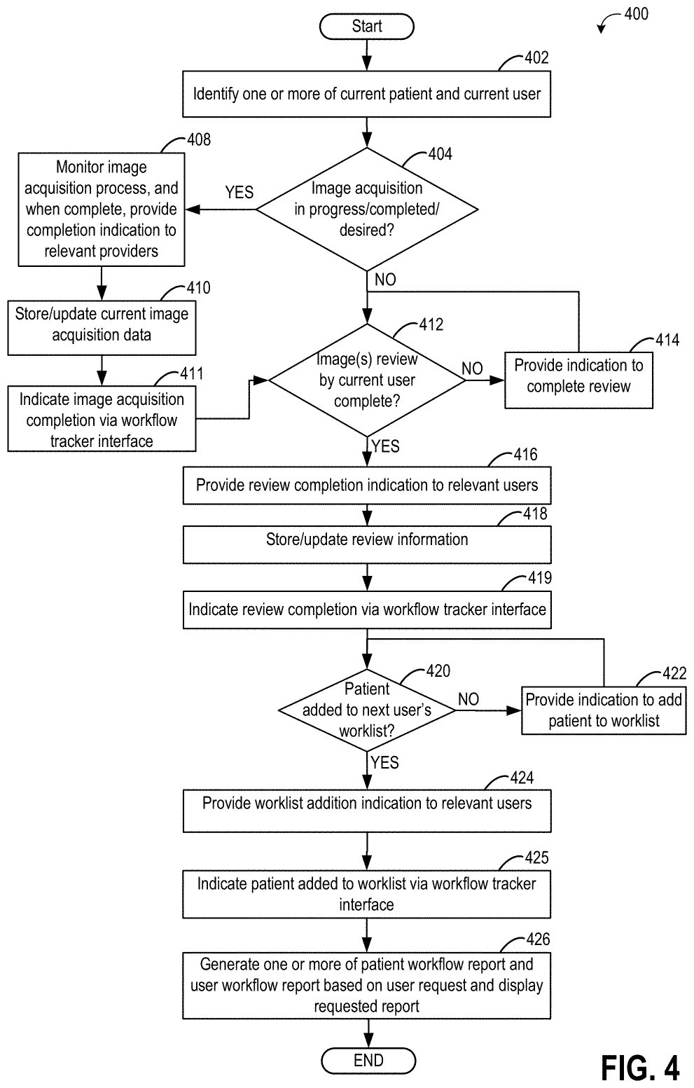

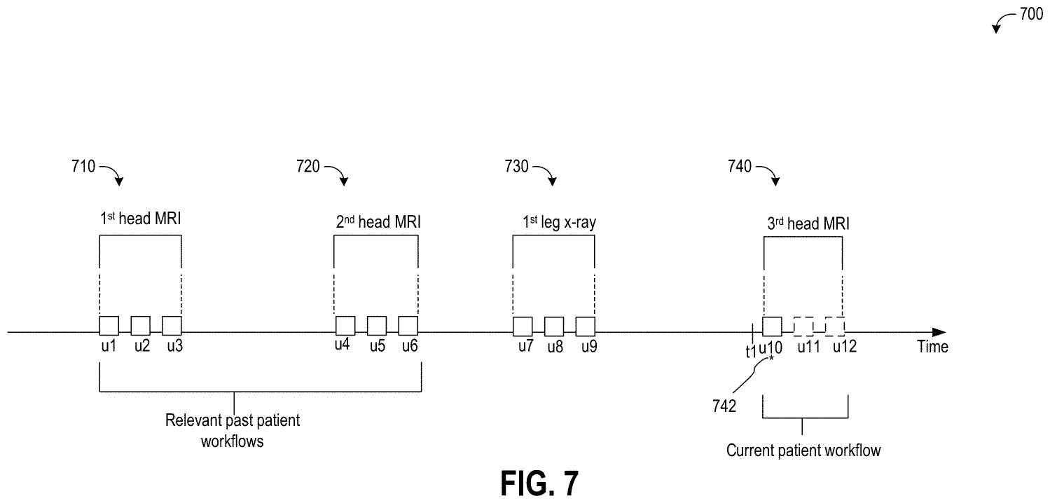

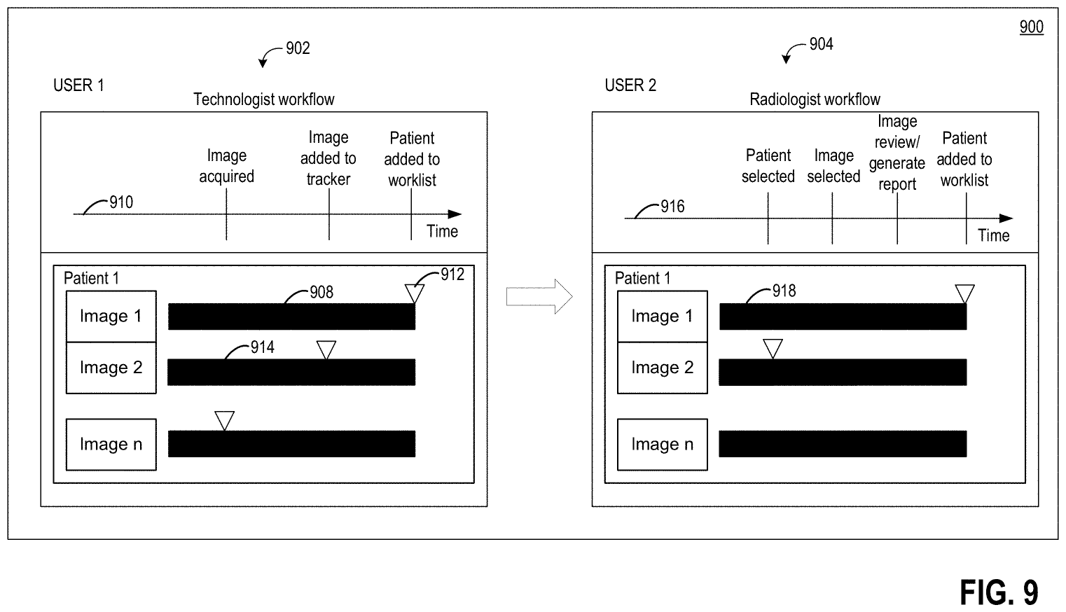

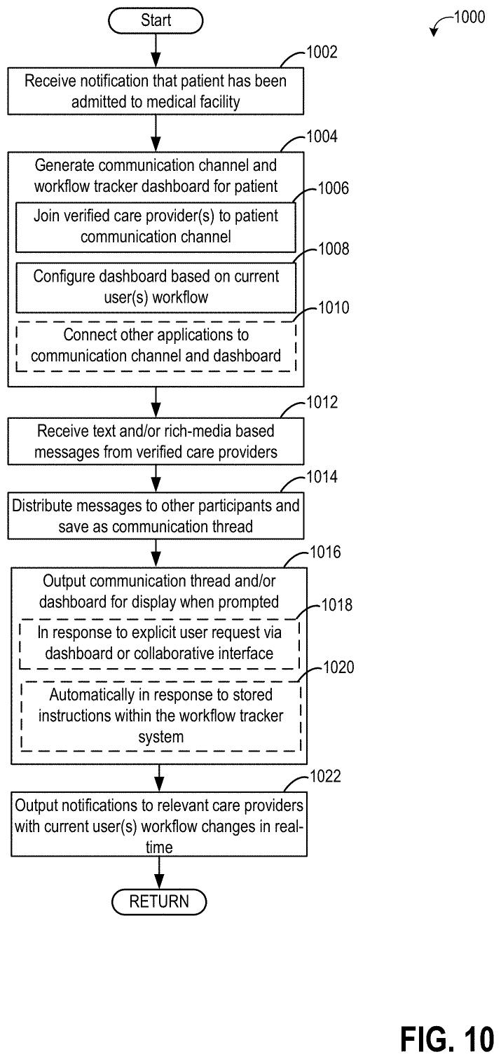

An example workflow tracker system is shown in FIG. 1. The workflow tracker system may be included in or associated with a medical facility and may include a communication channel comprising a communication thread and a dashboard for the tracked workflow of each care provider administering to a patient admitted to the medical facility. FIG. 2 shows an example of a workflow tracker system infrastructure that may facilitate user access to the patient's current medical information (updated in real-time) and previous medical records. The workflow tracker system may track the real-time workflow of the patient and/or user(s) and next step workflow recommendations may be output according to the methods illustrated in FIGS. 4, 5, 6, 8, and 10. FIGS. 7 and 9 show examples of timelines that may generated and utilized by the workflow tracker system to track the workflow of the patient and users in real-time. FIG. 10 illustrates a method for facilitating communication amongst and outputting notifications to collaborative users and previous care providers based on the tracked patient workflow. The real-time tracked workflow of collaborative care providers and the patient, as well as patient-specific medical information and recommendations for next workflow steps, may be displayed to the care providers and/or other users via a user interface on an exemplary viewer interface as shown in FIGS. 3A, 3B, 11, 12, and 13.

FIG. 1 schematically shows an example of a workflow tracker system 100 that may be implemented in a medical facility such as a hospital. The workflow tracker system 100 may include a collaborative space server system 102 and a workflow tracker server system 152. Collaborative space server system 102 may include resources (e.g., memory 130, processor(s) 132) that may be allocated to store and execute a communication thread, a dashboard, and a digital twin for each of a plurality of patients. For example, as shown in FIG. 1, a communication thread 104, dashboard 106, and digital twin 108 are stored on the collaborative space server system 102 for a first patient (patient 1); a plurality of additional communication threads, dashboards, and digital twins may be stored on server system 102, each corresponding to a respective patient (patient 2 up to patient N).

As explained above, the communication thread 104 may facilitate communication among a care provider team (which may include multiple care providers that are each providing care to the patient (e.g., patient 1)). Messages sent on the communication thread 104 may be saved and may be accessible via the dashboard 106 (and the dashboard may be accessible via the communication thread). Further, the patient medical information, including medical history, current state, vital signs, and other information, may be entered to the digital twin 108, which may be used to gain situational awareness, clinical context, and medical history of the patient to facilitate predicted patient states, procurement of relevant treatment guidelines, patient state diagnoses, etc.

Communication occurring on communication thread 104 may be displayed on one or more suitable display devices associated with a respective care provider device and/or medical facility administration device. Likewise, dashboard 106 may be displayed on the one or more display devices. As shown in FIG. 1, a plurality of care provider devices, from a first care provider device 134, a second care provider device 136, and on up to an nth care provider device 138, may be communicatively coupled to the collaborative space server system 102 and the workflow tracker server system 152. Each care provider device may include a processor, memory, communication module, user input device, display (e.g., screen or monitor), and/or other subsystems and may be in the form of a desktop computing device, a laptop computing device, a tablet, a smart phone, or other device. Each care provider device may be adapted to send and receive encrypted data and display medical information, including medical images in a suitable format such as digital imaging and communications in medicine (DICOM) or other standards. The care provider devices may be located locally at the medical facility (such as in a nurses station or in the room of a patient) and/or remotely from the medical facility (such as a care provider's mobile device).

When viewing communication thread 104 and/or dashboard 106 via a display of a care provider device, a care provider may enter input (e.g., via the user input device, which may include a keyboard, mouse, microphone, touch screen, stylus, or other device) that may be processed by the care provider device and sent to the server system 102. In examples where the user input is a message to be sent to other care providers, the message may be sent to the server system 102, where the message may be saved as part of the communication thread 104 and then the collaborative space server system 102 may send the message to other verified participants on the communication channel (e.g., the other care providers and/or one or more virtual healthcare assistants that are joined to the communication channel). In examples where the user input is a selection of a link or user interface control button of the dashboard, the user input may trigger display of the communication thread, trigger progression to a desired state of the dashboard (e.g., trigger display of desired patient medical information), trigger updates to the configuration of the dashboard, or other actions.

Collaborative space server system 102 includes a communication module 128, memory 130, and processor(s) 132 to store and execute the communication channel-dashboard pairs, and digital twins, as well as send and receive communications, graphical user interfaces, medical data, and other information. Communication module 128 facilitates transmission of electronic data within and/or among one or more systems. Communication via communication module 128 can be implemented using one or more protocols. In some examples, communication via communication module 128 occurs according to one or more standards (e.g., Digital Imaging and Communications in Medicine (DICOM), Health Level Seven (HL7), ANSI X12N, etc.). Communication module 128 can be a wired interface (e.g., a data bus, a Universal Serial Bus (USB) connection, etc.) and/or a wireless interface (e.g., radio frequency, infrared, near field communication (NFC), etc.). For example, communication module 128 may communicate via wired local area network (LAN), wireless LAN, wide area network (WAN), etc. using any past, present, or future communication protocol (e.g., BLUETOOTH™, USB 2.0, USB 3.0, etc.).

Memory 130 one or more data storage structures, such as optical memory devices, magnetic memory devices, or solid-state memory devices, for storing programs and routines executed by processor(s) 132 to carry out various functionalities disclosed herein. Memory 130 may include any desired type of volatile and/or non-volatile memory such as, for example, static random access memory (SRAM), dynamic random access memory (DRAM), flash memory, read-only memory (ROM), etc. Processor(s) 132 may be any suitable processor, processing unit, or microprocessor, for example. Processor(s) 132 may be a multi-processor system, and, thus, may include one or more additional processors that are identical or similar to each other and that are communicatively coupled via an interconnection bus.

The collaborative space server system 102 may be communicatively coupled to the workflow tracker server system 152 (e.g., via network 154 or another interface) and both server systems may be communicatively coupled to a network 154 as well as an infrastructure including one or more systems as shown in FIG. 2. As further shown in FIG. 1, a radiologist workflow module 140, a technologist workflow module 142, and a clinician workflow module 144 are stored on the workflow tracker server system 152 for a first patient (patient 1); a plurality of additional radiologist workflow modules, technologist workflow modules, and clinician workflow modules may be stored on the workflow tracker server system 152, each corresponding to a respective patient (patient 2 up to patient N). In some examples, the workflow tracker server system 152 may include additional workflow modules for other patient care providers, with each additional module corresponding to a respective patient. The workflow modules may receive and output data related to the current workflow of the radiologist, technologist, and clinician in real-time (e.g., the workflow of the user may be tracked in real-time by the workflow modules). In some embodiments, the workflow tracker server system 152 may include a patient workflow module for each patient (e.g., patient 1 up to patient N) corresponding to each respective user (e.g., user 1 up to user N). For example, user 1 (e.g., a radiologist) may have three patient workflow modules stored on the workflow tracker server system 152, with each workflow module receiving/outputting data related to the workflow of user 1 in real-time for each of three respective patients.

The systems and methods herein are with described with respect to user workflows that include radiological imaging but the methods disclosed may be implemented in virtually any other type of user workflow without departing from the scope of this disclosure. For example, in some embodiments, the workflow tracker server system 152 may not include the radiologist workflow module 140. In some embodiments, the workflow tracker server system 152 may include one or multiple physical therapist workflow modules, nurse workflow modules, surgeon workflow modules, ultrasound technician workflow modules, dietitian workflow modules, occupational therapist workflow modules, and so on.

In some examples, the workflow modules may receive workflow input from one or more medical devices and/or computing devices to determine a current phase of the workflow for a radiologist, a technologist, a clinician, and/or another user in real-time. The one or more medical devices and/or computing devices may be in the exam environment or outside the exam environment and communicatively coupled to the workflow tracker server system 152 via the network 154. In some examples, the one or more medical devices and/or computing devices may be directly communicatively coupled to the workflow tracker server system 152. For example, the workflow input may be output by a magnetic resonance imaging (MRI) machine and/or a device of a MRI system, such as a device housing a processor and memory. Additionally or alternatively, the workflow input may be output from a recording device or other device that logs events occurring in a workflow of a medical procedure as well as the workflow of the care provider(s). In some examples, the events may be logged via direct input by the care provider. For example, a user may enter input via a keyboard/touch screen, for example, indicating imaging has started, a findings report has been generated, and the patient has been added to a worklist, and so on. In another example, a visual sensor (e.g., a camera) may send data to the workflow module(s) to indicate the current event of the procedure and the user workflow. In some examples, the workflow modules may be linked to global positioning system (GPS) applications installed on care provider devices so that movement of the care providers within and outside of the medical facility may be tracked in real-time (e.g., the workflow tracker system may determine if the clinician is in a first exam room, a second exam, a third exam, outside the facility, etc.).

Similar to the collaborative space server system 102, the workflow tracker server system 152 includes a communication module 146, memory 150, and processor(s) 148 to store user workflow input and information, generate patient worklists, and next steps for a user (e.g., the technologist, the radiologist, the clinician) based on the current workflow of the patient and the user, as well as send and receive communications, graphical user interfaces, medical data, and other information. As previously described, the communication module 146 facilitates transmission of electronic data within and/or among one or more systems. Communication via communication module 146 can be implemented using one or more protocols. Memory 150 may include one or more data storage structures, such as optical memory devices, magnetic memory devices, or solid-state memory devices, for storing programs and routines executed by processor(s) 148 to carry out various functionalities disclosed herein. Processor(s) 148 may be any suitable processor, processing unit, or microprocessor, for example. Processor(s) 148 may be a multi-processor system, and, thus, may include one or more additional processors that are identical or similar to each other and that are communicatively coupled via an interconnection bus.

As used herein, the terms “sensor,” “system,” “unit,” or “module” may include a hardware and/or software system that operates to perform one or more functions. For example, a sensor, module, unit, or system may include a computer processor, controller, or other logic-based device that performs operations based on instructions stored on a tangible and non-transitory computer readable storage medium, such as a computer memory. Alternatively, a sensor, module, unit, or system may include a hard-wired device that performs operations based on hard-wired logic of the device. Various modules or units shown in the attached figures may represent the hardware that operates based on software or hardwired instructions, the software that directs hardware to perform the operations, or a combination thereof.

“Systems,” “units,” “sensors,” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations.

One or more of the devices described herein may be implemented over a cloud or other computer network. For example, collaborative space server system 102 is shown in FIG. 1 as constituting a single entity, but it is to be understood that the collaborative space server system 102 and/or the workflow tracker system 152 may be distributed across multiple devices, such as across multiple servers.

While not specifically shown in FIG. 1, additional devices described herein (care provider device 134, care provider device 136, and care provider device 138) may likewise include user input devices, memory, processors, and communication modules/interfaces similar to communication module 128, memory 130, and processor(s) 132 described above, and thus the description of communication module 128, memory 130, and processor(s) 132 likewise applies to the other devices described herein. As an example, the care provider devices (e.g., care provider device 134) may store user interface templates in memory that include placeholders for relevant information stored on server system 102. For example, care provider device 134 may store a user interface template for a workflow tracker interface that a user of care provider device 134 may configure with placeholders for desired patient information. When the workflow tracker is displayed on the care provider device, the relevant patient information may be retrieved from the collaborative space server system 102, the workflow tracker server system 152, and inserted in the placeholders. The patient information may include patient images, alerts, or other information, as explained in more detail below. The user input devices may include keyboards, mice, touch screens, microphones, or other suitable devices.

FIG. 2 is a block diagram of an example healthcare information infrastructure 200 that may be communicatively coupled to the workflow tracker server system 152 and the collaborative space server system 102 of FIG. 1. Coupling of the workflow tracker server system 152 and the collaborative space server system 102 to infrastructure 200 may facilitate workflow tracker user access to patient information/records/imaging results, current/previous care provider information, documents/reports, medication management, scheduling, and so on. Thus, the user may easily find current (in real-time) and previous information related to patient care from collaborative care providers/users as well as identify where the information came from thereby saving time and increasing operational efficiency. The infrastructure 200 includes a hospital information system (HIS) 204, a radiology information system (RIS) 206, a picture archiving and communication system (PACS) 208, an interface unit 210, a data center 212, and a workstation 214.

In the illustrated example, the HIS 204, the RIS 206, and the PACS 208 are housed in a healthcare facility and locally archived. However, in other implementations, the HIS 204, the RIS 206, and/or the PACS 208 can be housed one or more other suitable locations. In certain implementations, one or more of the PACS 208, RIS 206, HIS 204, etc., can be implemented remotely via a thin client and/or downloadable software solution. Furthermore, one or more components of the infrastructure 200 can be combined and/or implemented together. For example, the RIS 206 and/or the PACS 208 can be integrated with the HIS 204; the PACS 208 can be integrated with the RIS 206; and/or the three example information systems 204, 206, and/or 208 can be integrated together. In other example implementations, the infrastructure 200 includes a subset of the illustrated information systems 204, 206, and/or 208. For example, the infrastructure 200 may include only one or two of the HIS 204, the RIS 206, and/or the PACS 208. Information (e.g., scheduling, test results, exam image data, observations, diagnosis, etc.) can be entered into the HIS 204, the RIS 206, and/or the PACS 208 by healthcare practitioners (e.g., radiologists, physicians, and/or technologists) and/or administrators before and/or after patient examination.

The HIS 204 stores medical information such as clinical reports, patient information, and/or administrative information received from, for example, personnel at a hospital, clinic, and/or a physician's office (e.g., electronic medical records, electronic health record system, personal health record system, etc.). The RIS 206 stores information such as radiology reports, radiology exam image data, messages, warnings, alerts, patient scheduling information, patient demographic data, patient tracking information, and/or physician and patient status monitors. Additionally, the RIS 206 enables exam order entry (e.g., ordering an x-ray of a patient) and image and film tracking (e.g., tracking identities of one or more people that have checked out a film). In some examples, information in the RIS 206 is formatted according to the HL-7 (Health Level Seven) clinical communication protocol. In certain examples, a medical exam distributor is located in the RIS 206 to facilitate distribution of radiology exams to a radiologist workload for review and management of the exam distribution by, for example, an administrator.

The PACS 208 stores medical images (e.g., x-rays, scans, three-dimensional renderings, etc.) as, for example, digital images in a database or registry. In some examples, the medical images are stored in the PACS 208 using the Digital Imaging and Communications in Medicine (DICOM) format. Images are stored in the PACS 208 by healthcare practitioners (e.g., imaging technologists, physicians, radiologists) after a medical imaging of a patient and/or are automatically transmitted from medical imaging devices to the PACS 208 for storage. In some examples, the PACS 208 can also include a display device and/or viewing workstation to enable a healthcare practitioner or provider to communicate with the PACS 208.

The interface unit 210 includes a hospital information system interface connection 216, a radiology information system interface connection 218, a PACS interface connection 220, and a data center interface connection 222. The interface unit 210 facilities communication among the HIS 204, the RIS 206, the PACS 208, and/or the data center 212. The interface connections 216, 218, 220, and 222 can be implemented by, for example, a Wide Area Network (WAN) such as a private network or the Internet. Accordingly, the interface unit 210 includes one or more communication components such as an Ethernet device, an asynchronous transfer mode (ATM) device, an 802.11 device, a DSL modem, a cable modem, a cellular modem, etc. In turn, the data center 212 communicates with the workstation 214, via a network 224, implemented at a plurality of locations (e.g., a hospital, clinic, doctor's office, other medical office, or terminal, etc.). The network 224 is implemented by, for example, the Internet, an intranet, a private network, a wired or wireless Local Area Network, and/or a wired or wireless Wide Area Network. In some examples, the interface unit 210 also includes a broker (e.g., a Mitra Imaging's PACS Broker) to allow medical information and medical images to be transmitted together and stored together. The network 224 may be an example of network 154 discussed at FIG. 1.

The interface unit 210 receives images, medical reports, administrative information, exam workload distribution information, and/or other clinical information from the information systems 204, 206, 208 via the interface connections 216, 218, 220. If necessary (e.g., when different formats of the received information are incompatible), the interface unit 210 translates or reformats (e.g., into Structured Query Language (“SQL”) or standard text) the medical information, such as medical reports, to be properly stored at the data center 212. The reformatted medical information can be transmitted using a transmission protocol to enable different medical information to share common identification elements, such as a patient name or social security number. Next, the interface unit 210 transmits the medical information to the data center 212 via the data center interface connection 222. Finally, medical information is stored in the data center 212 in, for example, the DICOM format, which enables medical images and corresponding medical information to be transmitted and stored together.

The medical information is later viewable and easily retrievable at the workstation 214 (e.g., by their common identification element, such as a patient name or record number). The workstation 214 can be any equipment (e.g., a personal computer) capable of executing software that permits electronic data (e.g., medical reports) and/or electronic medical images (e.g., x-rays, ultrasounds, MRI scans, etc.) to be acquired, stored, or transmitted for viewing and operation. Thus, workstation 214 may be an acquisition workstation, a clinical viewer workstation, an administrative workstation in a clinical and/or hospital environment, a laboratory workstation, or a device utilized for collaborative medical image/data viewing, etc. It will be appreciated that the infrastructure 200 may include any number of workstations.

The workstation 214 receives commands and/or other input from a user via, for example, a keyboard, mouse, track ball, microphone, etc. The workstation 214 is capable of implementing a user interface 226 (also referred to herein as a workstation interface) to enable a healthcare practitioner and/or administrator to interact with the infrastructure 200. For example, in response to a request from a physician, the user interface 226 presents a patient medical history. In other examples, a radiologist is able to retrieve and manage a workload of exams distributed for review to the radiologist via the user interface 226. In further examples, an administrator reviews radiologist workloads, exam allocation, and/or operational statistics associated with the distribution of exams via the user interface 226. In some examples, the administrator adjusts one or more settings or outcomes via the user interface 226.

The workstation 214 may be configured to or capable of communicating with one or more servers including server 228, workflow tracker server 152, and collaborative space server 102. The workstation 214 may include a processing unit, a non-transitory memory, and/or an image display monitor, for example. The workstation interface 214 may be implemented as a network card connecting to a TCP/IP based network, but may also be implemented as a parallel port interface, for example.

The workstation 214 may retrieve or receive data from the one or more servers for display to one or more users. For example, the workstation 214 may retrieve or receive image data representative of a computed radiography (“CR”) image of a patient's chest. A radiologist or user may then examine the image for any objects of interest, such as tumors, lesions, etc., for example. The workstation interface 226 may include a workflow tracker interface 275 for enabling the user to retrieve data from and store data in one or more of workflow tracker server system 152 and collaborative space server system 102. The workflow tracker interface 275 may display workflow progress of one or more of a patient and one or more healthcare providers involved in the workflow of the patient. Further, when requested, the workflow tracker interface may display information regarding various workflow steps in the workflow of the patient, as further discussed below.

A workflow tracker application may be launched via the workflow tracker interface 275 within the workstation interface 226. When launched, the workflow tracker application may execute one or more methods stored in non-transitory memory of the workflow tracker server system 152 to track one or more workflows in real-time including patient workflows and associated workflows of healthcare providers (also, referred to herein as user workflows). Details of the workflow tracker interface 275, and the various methods for tracking the one or more workflows are described below with respect to FIGS. 3-13.

The example data center 212 of FIG. 2 is an archive to store information such as images, data, medical reports, and/or, more generally, patient medical records. In addition, the data center 212 can also serve as a central conduit to information located at other sources such as local archives, hospital information systems/radiology information systems (e.g., the HIS 204 and/or the RIS 206), or medical imaging/storage systems (e.g., the PACS 208 and/or connected imaging modalities). That is, the data center 212 can store links or indicators (e.g., identification numbers, patient names, or record numbers) to information. In the illustrated example, the data center 212 is managed by an application server provider (ASP) and is located in a centralized location that can be accessed by a plurality of systems and facilities (e.g., hospitals, clinics, doctor's offices, other medical offices, and/or terminals). In some examples, the data center 212 can be spatially distant from the HIS 204, the RIS 206, and/or the PACS 208 (e.g., at GENERAL ELECTRIC® headquarters).

The example data center 212 of FIG. 2 includes a server 228, a database 230, and a record organizer 232. The server 228 receives, processes, and conveys information to and from the components of the infrastructure 200. The database 230 stores the medical information described herein and provides access thereto. The example record organizer 232 of FIG. 2 manages patient medical histories, for example. The record organizer 232 can also assist in procedure scheduling, for example.

Certain examples can be implemented as cloud-based clinical information systems and associated methods of use. An example cloud-based clinical information system enables healthcare entities (e.g., patients, clinicians, sites, groups, communities, and/or other entities) to share information via web-based applications, cloud storage and cloud services.

FIG. 3A shows an example display device 302 displaying an embodiment of a workflow tracker interface 275 of a workflow tracker system, such as workflow tracker server system 152 at FIG. 1. The workflow tracker interface 275 may be displayed as part of a workstation interface, such as workstation user interface 226 at FIG. 2. In one example, the workflow tracker interface 275 may be displayed as part of one or more of an acquisition interface of an acquisition workstation communicatively coupled to an imaging modality, a clinical viewer interface of a clinical workstation (e.g., PACS workstation), a healthcare interface of a healthcare administrative workstation, and any workstation interface that is utilized to access and update a patient information. Further, the workflow tracker interface 275 may be displayed as part of a device interface of a care provider device (e.g., care provider device 134 of FIG. 1, a smart phone, a tablet) or another suitable device.

The workstation interface 275 may be utilized to display one or more of patient information based on the current and previous medical visits including a patient workflow, and one or more real-time user workflows of one or more users (healthcare and/or collaborative care providers) involved in the patient workflow. The workstation interface 275 may include one or more command buttons that may allow a user to review acquired radiological images of the patient, view acquisition information for the acquired radiological images, view/edit/update a current patient worklist, to generate finding(s) reports, and track the workflow of collaborative users in real-time. In particular, the workstation interface 275 may include an Images Taken control button 310, an Added to Worklist control button 312, an Image Review control button 314, and a Generate Report control button 316.

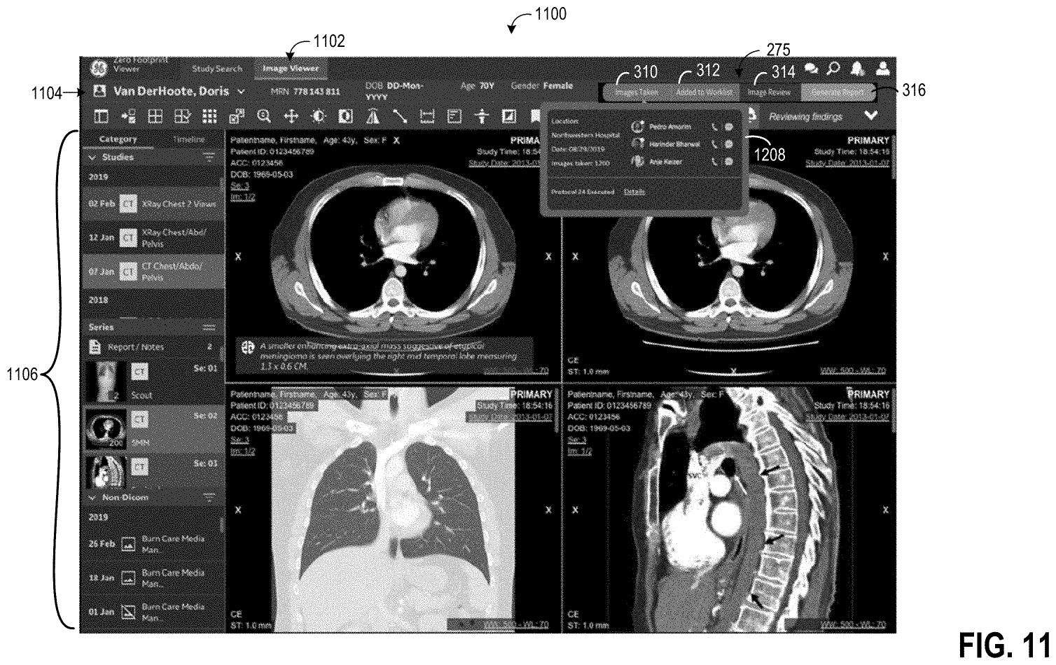

When selected, the Image Review control button 314 may allow the user to select and review patient images acquired during the workflow of the current users as well as images acquired by previous care providers, as further shown and described with respect to FIG. 11.

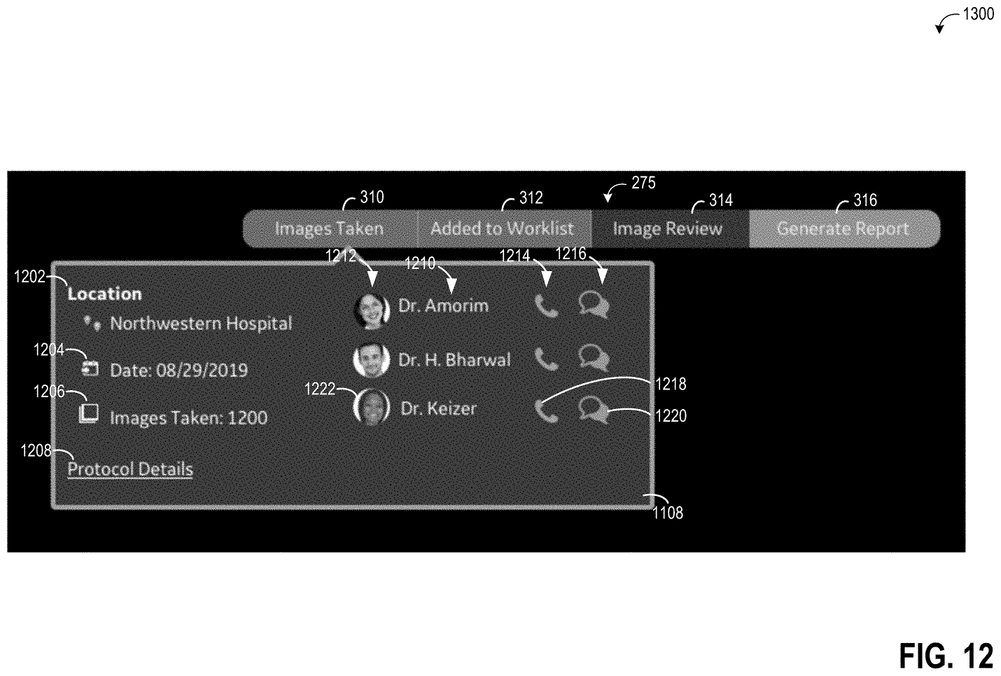

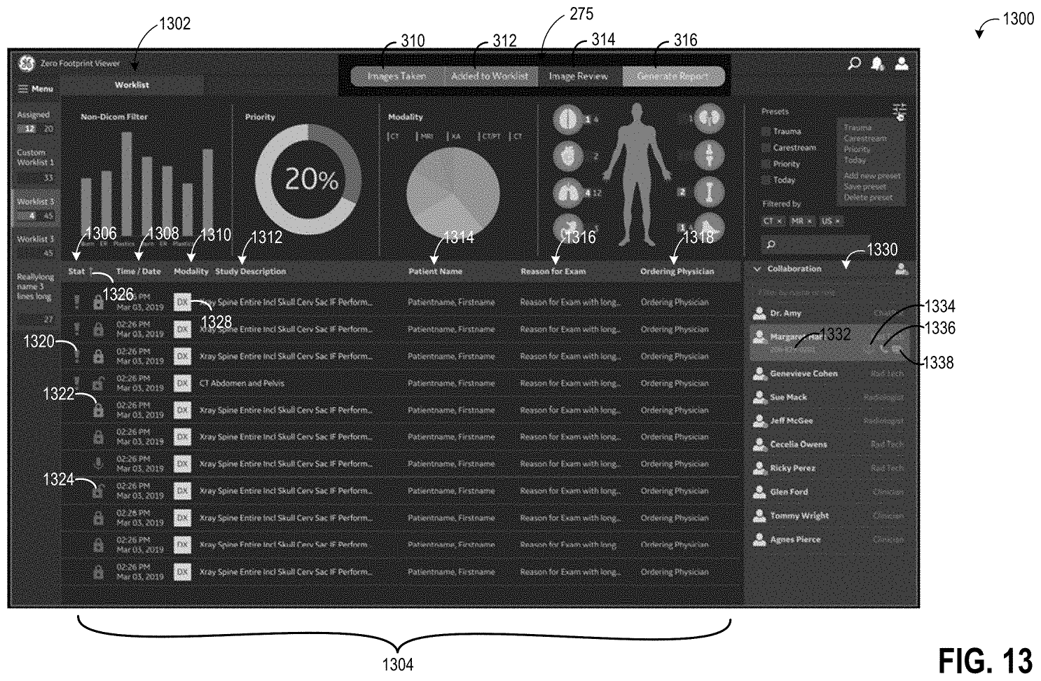

The Images Taken control button 310 may be used to display acquisition information for any selected patient image, as further described with respect to FIG. 12. A patient worklist may be displayed after selection of the Added to Worklist control button 312, as further described with respect to FIG. 13. Upon selection, the Generate Report control button 316 may allow the user to generate a report for a patient based on the user's current findings, a report of other collaborative user(s) findings, and/or of findings from previous care providers of the patient as further described below. In some examples, the Generate Report control button 316, when selected, may display a pull down menu including a generate report for a patient workflow option and a generate report for current user workflow option. Thus, a user may obtain a patient workflow report and/or a user workflow report via the workflow tracker interface 275.

In one example, the workflow interface 275 may provide visual tracking of a user's workflow. Specifically, the user's current workflow step may be highlighted in the workflow tracker interface 275 with a first color highlight, the user's completed workflow step may be highlighted in the workflow tracker interface 275 with a second color highlight, and the user's next workflow step may be highlighted in the workflow tracker interface 275 with a third color highlight (the third color different from the first color and the second color highlights). An exemplary embodiment of a workflow tracker interface 318 illustrating visual tracking of the user's workflow is shown at FIG. 3B. The workflow tracker interface 318 shows four workflow steps including an image taken step by control button 320, an added to worklist step by control button 322, an image review step by control button 324, and a generate report step by control button 326. In the present example, the user may have completed the images taken and the added to worklist steps, and may be currently reviewing the images taken. As such, control buttons 320 and 322 (corresponding to image taken and added to worklist steps) are indicated by a first highlight color (e.g., grey), control button 324 corresponding to current step of image review is indicated by a second highlight color (e.g., darker grey), and control button 328 corresponding to future step of generate report is indicated by a third highlight color (e.g., light grey). If the user completes image review, and moves to generate report step, then control buttons 320, 322, and 324 may be indicated by the first color (e.g., grey) and the control button 326 corresponding to current step may be indicated by second color (e.g., darker grey). In this way, the workflow interface provides visual tracking of the user's workflow.

In some examples, the workflow interface 318 may enable visual tracking of a patient's overall workflow, and thus, the control buttons 320, 322, 324, and 326 may correspond to a patient's workflow. Further, an additional indication as to whether the workflow tracker interface 318 shows a patient's workflow or a user workflow may be displayed. As an example, a worklist of for a user may include a first patient and a second patient. For the first patient, the image taken and the added to worklist may be completed (either by the user or a different user), and as such, the control buttons 320 and 322 may be indicated by the first color highlight (e.g., grey). Further, for the first patient, the user may be in the process of reviewing the images, and thus, the control button 324 may be indicated by the second color highlight (e.g., darker grey). The future workflow step may be indicated by the third color highlight (e.g., lighter grey). When the user switches to the second patient in the user's worklist, the visual indications on the workflow interface 318 may change depending on the workflow of the second patient. For example, for the second patient, if the workflow steps of images taken, added to worklist, and image review are completed, and the generate report is pending, control buttons 320, 322, and 324 may be indicated by the first color (e.g., grey) and the control button 326 corresponding to pending (not current) step may be indicated by third color (e.g., lighter grey). In this way, the workflow interface provides visual tracking of the patient's workflow. Although not depicted, an additional indication on the workflow tracker interface 318 may be provided to indicate whether the workflow tracker interface is showing the patient's workflow or the user's workflow.

While the above examples are illustrated with respect to different shades of grey, it will be appreciated that different shades of any color may be utilized for visual indications on the workflow interface. Also, different colors with corresponding color legends may be utilized to visually indicate the different stages of workflow, and are within the scope of the disclosure.

Referring back to FIG. 3A, in some embodiments, the workflow interface 275 may include additional control buttons. For example, the workflow interface 275 may include a Future Steps control button that may inform users of algorithm predicted next steps based on the collaborative workflow of all users via a generated list upon selection. In some examples, the workflow interface 275 may include a User Timeline control button. Upon selection, the User timeline button may generate a list of current users. The list may be selectable to launch a timeline that includes past and future steps for each current user. In some examples, an overview timeline for the patient that includes all the workflow for all current and/or previous users may be generated. In some examples, the workflow interface 275 may include a Current User Location control button that may allow collaborative users to the track the whereabouts of other current users in real-time. In some examples, the workflow interface 275 may include a Message Board control button that launches a communication channel/board for all verified care providers (e.g., current and previous care providers) of the patient upon selection. The communication channel/board may allow the verified care providers to communicate in real-time via text- and/or rich-media based messages during the current user(s) workflow and as patient information is updated in real-time. In some examples, upon launching the workflow interface 275, notifications of updates to the patient worklist may be automatically output to all current users (e.g., the notifications may be output to a linked mobile device, a care provider device, or another suitable digital display). Thus, the workflow interface 275 may provide an interactive and visual means for tracking the workflow of one or more collaborative and healthcare care providers (e.g., a radiologist, a cardiologist, a clinician) for a respective patient in real-time. Further, the workflow interface 275 may provide the user with detailed information regarding past steps taken with regard to patient care as well as predicted future steps based on a tracked patient workflow (past and current).

As discussed above, a workflow tracker application may be launched on one or more of an acquisition interface and a viewer interface, with the workflow tracker interface 275 providing visually tracking, enabling interaction with the user, providing requested information, and providing one or more recommendations to the user. Launching of the workflow tracker application may enable communication, via a communication network (such as network 224 at FIG. 2 or network 154 at FIG. 1), between a workstation (such as workstation 226 at FIG. 2) and the workflow tracker server system. Further, when the workflow tracker application is launched, one or more methods, including method 400, 500, 600, 800, and 1000 may be executed via a processor of a workflow tracker system, such as the workflow tracker system 152 at FIG. 1. The methods 400, 500, 600, 800, and 1000 are discussed further below with respect to FIGS. 4, 5, 6, 8, and 10 respectively.

FIG. 4 shows a high-level flowchart illustrating an example method 400 for updating one or more of an image acquisition information, a patient information, and a review information to a workflow tracker module for a patient, such as at least one of patient workflow tracker modules 151 at FIG. 1. Specifically, the method 400 may update the one or more acquisition, patient, and review information for the patient with respect to a current user. The method 400 may be implemented by a processing system, such as controller 148 at FIG. 1, an edge device connected to the processing system, a cloud in communication with the processing system, or any appropriate combination thereof. The method 400 is described with regard to the systems and components of FIGS. 1, 2, 3A, and 3B, although it should be appreciated that method 400 may be implemented with other systems and components without departing from the scope of the present disclosure. The method 400 may be executed in response to launching a workflow tracker application (alternatively referred to as workflow tracker) on a workstation, such as workstation 214 at FIG. 2. Method 400 may include tracking a patient workflow in real-time, and outputting a continually updated timeline of the workflow via a workflow user interface, such as workflow interface 275 at FIGS. 2 and 3A.

The method 400 and other methods herein will be described with respect to a medical imaging workflow for the patient. The workflow tracker may be adapted for any clinical workflow for the patient, and is within the scope of the disclosure. Further, while the method 400 and the other methods herein will be described below with respect to launching the workflow tracker application in an acquisition workstation and/or a clinical viewer workstation, it will be appreciated the workflow tracker application may be implemented and launched with respect to any workstation, such as an administrative workstation in a clinical and/or hospital environment, a laboratory workstation, a device utilized for collaborative medical image/data viewing, etc.

Method 400 begins at 402. At 402, the method 400 includes identifying one or more of a current patient and a current user. In one example, a user may launch an acquisition interface or a clinical viewer interface (also referred to herein as medical image viewer interface) on a workstation, such as an acquisition workstation of an imaging modality (e.g., MRI system, x-ray imaging system, etc.) or a clinical workstation communicatively coupled to an information/data system (e.g., the PACS, HIS, RIS, etc.). The workflow tracker application may be launched in response to the user launching the corresponding workstation interface, and the user may be identified based on user information entered to access the interface. For example, the user may log-in to launch the acquisition interface or the clinical viewer interface, and the workflow tracker application may utilize the user log-in information (name, clinical role, etc.) to identify the user. In some examples, in addition to or alternative to workstation interface log-in, the workflow tracker application may request user to log in, via a workflow tracker interface, such as workflow tracker interface 275 at FIGS. 2 and 3A, and the user information may be obtained from the workflow tracker log in information.

The current patient may be identified based on the user selecting the patient from a worklist. For example, upon launching an acquisition interface or a viewer interface, a corresponding patient worklist for the user may populated and provided to user via the acquisition or the viewer interface. In response to the user selecting a patient from the worklist, the current patient may be identified.

Next, method 400 proceeds to 404. At 404, the method 400 includes evaluating a first stage in the imaging workflow. In the present example, the first stage is an image acquisition process. According, at 404, the method 400 includes determining if image acquisition is desired, in progress, or completed. For example, if the acquisition interface is launched on the acquisition workstation of an imaging modality, the workflow tracker (that is also launched with the acquisition interface) may determine that the image acquisition is desired, in progress, or completed. In some examples, the workflow tracker may output an indication, via the workstation interface for example, to the user requesting if image acquisition is desired during the current session by the current user. Thus, if the acquisition interface is launched and/or the user indicates that imaging is desired, the answer at 404 is YES, and the method 400 proceeds 408.

At 408, the method 400 includes monitoring the image acquisition process, and when image acquisition is complete, the method 400 may provide an indication to one or more relevant team members that the image acquisition is complete. In one example, image acquisition completion may be determined based on user indication of completion on one or more of the acquisition interface and the workflow tracker application. In some examples, the imaging modality may include one or more sensors, such as vision sensors, for monitoring acquisition progression, and as such, a processor coupled to the imaging modality may provide an indication to the workflow tracker application, based on the one or more sensors, that the acquisition is complete.

Further, the indication may be one or more of a text message to a corresponding personal computing device (e.g., mobile device, laptop, desktop, tablet, etc.) of one or more relevant team members, an email message to the one or more relevant team members, and a notification on an application interface of the corresponding personal computing devices (e.g., a mobile workflow tracker application on the mobile device) of the one or more relevant team members. The one or more team members may include one or more healthcare professionals and collaborative care professions involved in one or more of image acquisition, image review, diagnosis, treatment plan, and follow-up for the patient. The one or more healthcare and collaborative care professionals may include one or more a technologist, radiologist, clinician, ordering physician, assistants, and members of a multidisciplinary team. In some examples, the patient may also receive notification as the patient moves through the imaging workflow.

Next, the method 400 proceeds to 410. At 410, the method 400 includes storing and/or updating current image data and current image acquisition data. In one example, the current acquisition data may be updated and stored on the workflow tracker sever while the acquired image data may be stored on another server, such as the PACS server, wherein each of the image data and the acquisition data is associated with a corresponding identifier that may be utilized for later retrieval.] In another example, current acquisition data may be integrated with the acquired image data. For example, current acquisition data may be embedded with each image scan data, and may be stored in the PACS server, or any reference server, which may be communicatively coupled to workflow tracker server such that when the workflow tracker application is executed, the acquisition data and the image data for a selected patient may be retrieved, when requested.

Further, the method 400 may update current user workflow information for the current user involved in the patient workflow via the workflow tracker. Thus, the workflow tracker tracks workflow of each user (that is, each healthcare/collaborative care professional) involved in the patient workflow.

The acquisition data may include acquisition parameters of the one or more images acquired. The acquisition parameters may include a time and date the images were acquired, the acquisition protocol used during the current acquisition, the modality employed (e.g., x-ray imaging, CT scanning, digital mammography), systematic parameters of the modality employed (e.g., scanning mode, temperature, current output, etc.), the body part imaged (e.g., chest, spine, pelvis, abdomen, etc.), the priority of imaging within the patient worklist (e.g., high priority, low priority, a number in a priority scale etc.), and a location where the images were acquired (e.g., the name and address of the medical facility). Further, acquisition data may include user information, including data regarding the user performing the imaging and clinical position (e.g., radiologist, technologist, physician, cardiologist, neurologist, surgeon etc.).

In this way, through the workflow tracker, tracking of one or more of image data, acquisition data, and user data, is enabled. Upon storing and/or updating image data and image acquisition data, method 400 proceeds to 411.

At 411, the method 400 includes indicating image acquisition completion via the workflow tracker interface. This includes providing a visual indication regarding image acquisition completion via an image taken control button, such as the control button 310 at FIG. 3A. The visual indication via workflow tracker interface for image acquisition completion may include a color highlight change of the image taken control button. For example, when the image acquisition is in progress, the image taken control button may be highlighted with a color or shade of a color visually indicating that the image acquisition is in progress. When the image acquisition is completed, the image taken control button may be highlighted with a different color or a different shade of the color visually indicating that the image acquisition is completed. If the image acquisition has not yet begun, the image taken control button may be highlighted with a second different color or a second different shade of the color. An exemplary visual tracking indicating workflow status (e.g., not yet started, in progress, and completed) via the workflow tracker is described at FIG. 3B. In this way, the method 400 may update the workflow user interface based on a status (e.g., not yet started, in progress, completed) the workflow step. Upon updating the workflow user interface appearance, the method 400 proceeds to 412.

Returning to 404, if image acquisition is not in progress, completed, or desired, answer at 404 is NO, and the method 400 proceeds to 412.

At 412, the method 400 includes confirming if a second stage in the imaging workflow is completed. In the present example, the second stage in the imaging acquisition, is an image review stage. Accordingly, at 412, the method 400 may include determining if the image review of the one or more acquired images is completed by the current user. In one example, the determination of image review completion may be determined based on user indication. In another example, in addition to or alternative to the user indication, the method 400 may monitor a review/annotation field on the corresponding interface (e.g., acquisition interface, clinical viewer interface, any other application interface that the user is utilizing, etc.) to determine if review by the current user is completed. For example, the method may completion of one or more of observations, initial impressions, diagnosis, findings, expected next steps, complete analysis, etc., of the acquired images, indicated by the user, in the review/annotation field of the acquisition interface, the clinical viewer interface, and/or the corresponding application with which the workflow tracking application is implemented

In some examples, the review information may be a report that includes relevant information about diagnosis, condition, response to therapy, results of a procedure performed based on the reviewed image(s), and/or user footnotes. For example, the review information may be a radiology report generated by a current user identified as the radiologist. The radiology report may include sections that describe the indication for the study/exam, details of the imaging procedure performed, a description of the results of the exam, the relationship of the results to other previous studies/clinical information, a conclusion of the findings of the exam, support for the conclusion, a summary of the overall diagnosis, and any footnotes to the report.

If the review is not complete, the method 400 proceeds to 414. At 414, the method 400 may output an indication, via the workflow tracker application interface, to the user to complete the review and/or confirm completion of review. The method then returns to monitor review completion.

If the review is complete, the answer at 412 is YES, and the method 400 proceeds to 416. At 416, the method 400 may provide an indication to one or more relevant team members that the image review by the current user is complete. The indication at 416 may be similar to the indication step at 408, and will not be repeated for brevity.

Next, method 400 proceeds to 418 to store and/or update review information. As discussed with respect to step 410, in one example, the review information may be updated and stored on the workflow tracker sever and may be associated with a review identifier that may be utilized for later retrieval. In another example, review data may be integrated with the acquired image data and acquisition data that are then stored in the PACS server, or any reference server, which may be communicatively coupled to workflow tracker server such that when the workflow tracker application is executed, the review data may be retrieved, via the workflow tracker application, when requested.

At 419, the method 400 includes indicating image review completion via the workflow tracker interface. This includes providing a visual indication regarding image review completion via an image review control button, such as the control button 314 at FIG. 3A. The visual indication via workflow tracker interface for image review completion may be similar to the visual indication for the image taken completion as discussed at 411. Briefly, the visual indication via workflow tracker interface for image review completion may include a color highlight change of the image review control button. For example, when the image review is in progress, the image review control button may be highlighted with a color or shade of a color visually indicating that the image acquisition is in progress. When the image review is completed, the image review control button may be highlighted with a different color or a different shade of the color visually indicating that the image acquisition is completed. If the image review has not yet begun, the image taken control button may be highlighted with a second different color or a second different shade of the color. Upon updating the workflow user interface appearance, the method 400 proceeds to 420.

Next, method 400 proceeds to 420. At 420, the method 400 includes evaluating a third stage of imaging workflow. In the present example, the third stage may include determining if the patient has been added to a next user's worklist. For example, if the current user is a technologist acquiring images, the method may determine if the patient has been added to a radiologist's workflow who is assigned to evaluate the images acquired by the technologist. If the current user is the radiologist, the method may determine if the patient has been added to the patient's clinician/expert/ordering physician for diagnosis confirmation and treatment plan.

In one example, the workflow tracker may be utilized for adding patients to one or more of a next user's worklist and a current user's worklist. Accordingly, the method 400 may determine that the patient is added to the next user's worklist based on current user's action (e.g., based on the current user clicking an add to worklist icon (discussed further below with respect to FIG. 11) on the workflow tracker interface, and the workflow tracker communicating and updating a next user's worklist on the corresponding user's workflow module)

In another example, upon completion of review, the method 400 may automatically identify the next user in the workflow, and add the patient to the next user's worklist

If the answer at 420 is NO, the method 400 proceeds to 422. At 422, the method 400 includes providing an indication to the current user, via the workflow tracker application interface, to add the patient to the next user's worklist. The method 400 then returns to monitor addition of patient to the worklist.

If the patient is added to next user's worklist, the answer at 420 is YES, and the method 400 proceeds to 424 to provide indication of addition of the patient to the worklist to one or more relevant team members. The indication may be similar to the indication provided at 408, and will not be repeated.

At 425, the method 400 includes indicating that the patient is added to the worklist via the workflow tracker interface. This includes providing a visual indication via an added to worklist control button, such as the control button 312 at FIG. 3A. The visual indication via workflow tracker interface for adding the patient to the worklist may include visually changing a color highlight of the worklist control button of the workflow interface. The visual indication for adding the patient to the worklist may be similar to the visual indication for the image taken completion as discussed at 411 and image review completion as discussed at 419, and will not be repeated again. Upon updating the workflow user interface appearance, the method 400 proceeds to 420.

Next, upon providing the indication of worklist addition to one or more relevant team members, the method 400 proceeds to 426. At 426, the method 400 may include generating one or more of a patient workflow report and a user workflow report based on user request. Specifically, the method 400 may generate one or more of the workflow reports based on request by the user via the workflow tracker application interface. For example, the workflow tracker application may include a generate report icon, and in response to the user clicking the generate report icon, the method 400 may generate one or more of the patient workflow report and the user workflow report. The patient workflow report may include a current imaging workflow for the patient including a list of acquired images, acquisition information for each of the current acquired images, collaboration information including communication information (e.g., phone number, chat interface, etc.) for each of the one or more team members for the imaging workflow, and a summary of one or more of priority of imaging, modality and imaging modes utilized, body parts images, and review. The current user workflow report may include imaging progression indication through different stages of the workflow tracker for the patient. Example user workflow reports are shown and described with respect to FIGS. 9A-9B. An example workflow tracker application interface including the generate report icon is shown and described with respect to FIGS. 3A and 3B.

In some examples, the user may request a past workflow report for the current patient. For example, the user may request, via the workflow tracker, to generate a timeline of various workflows associated with the patient. When the patient workflow timeline is requested, the workflow tracker may identify all the workflow data stored for the patient at one or more of the workflow tracker server, PACS, HIS, RIS, etc., and generate a comprehensive workflow report.

Further, at 426, the method 400 includes displaying one or more of the reports generated to the user, via the respective workstation interface within which the workflow tracker application is executed. For example, if the user is utilizing the acquisition workstation, and the acquisition interface is launched with the workflow tracker application, the generated report may be displayed on the acquisition interface. If the user is utilizing a PACS workstation and the clinical viewer interface is launched with the workflow tracker application, the generated report may be displayed on the viewer interface.

Further, a status of report generation may be indicated via the workflow tracker interface. This includes providing a visual indication via a generate report control button, such as the control button 316 at FIG. 3A. The visual indication via workflow tracker interface may include visually changing a color highlight of the generate report control button of the workflow interface based on the status (e.g., not yet started, in progress, and completed) of the report generation. The visual indication for generating report may be similar to the visual indication for the image taken completion as discussed at 411 and image review completion as discussed at 419, and will not be repeated again.

Further, it will be appreciated that one or more of the patient workflow report and the user workflow report may be generated at any time based on user request via the workflow tracker application interface.

The method 400 then ends.

In this way, the workflow tracker application may be utilized to monitor current/identify current imaging workflow step, and store and/or update information based on the current imaging workflow. While the method 400 describes the first stage as the imaging stage, the second stage as image review stage, and the third stage as adding the patient to the next user's worklist, the order of the different stages are interchangeable, and within the scope of the disclosure.

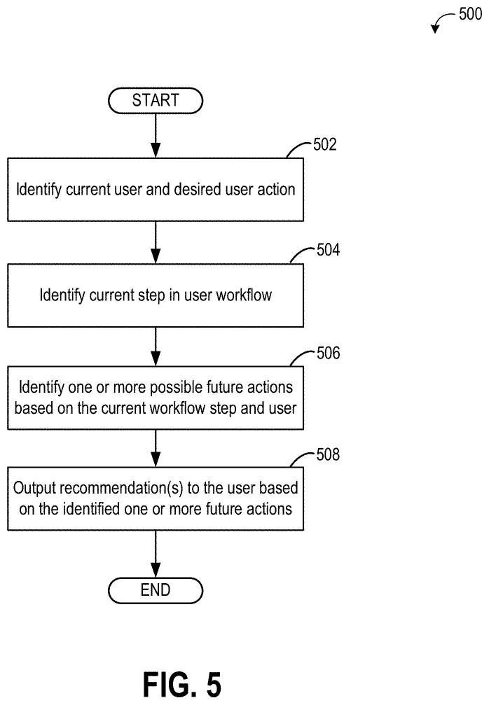

FIG. 5 is a high-level flow chart showing an exemplary method 500 for tracking a current workflow for the user in an imaging workflow of the patient, and providing recommendations based on user status and expected workflow steps for the user during the current workflow via the workflow tracker application. The method 500 may be implemented by a processing system, such as controller 148 at FIG. 1, an edge device connected to the processing system, a cloud in communication with the processing system, or any appropriate combination thereof. The method 500 is described with regard to the systems and components of FIGS. 1, 2, 3A, and 3B, although it should be appreciated that method 500 may be implemented with other systems and components without departing from the scope of the present disclosure. The method 500 may be executed in response to launching the workflow tracker application on a workstation, such as workstation 214 at FIG. 2.

Method 500 begins at 502. At 502, the method 500 includes identifying a current user and a desired user action. The user may be one of a group of collaborative care and healthcare providers administering care to an admitted patient. Identifying current user at step 502 may be similar to user identification discussed at step 402, and thus, will not be repeated. Further, at 502, the method 500 may include determining a desired user action based on the current user identification and based on each user's typical workflow. For example, a desired user action for a technologist may include image acquisition, a first review comprising one or more initial findings from the acquired images, and adding patient to a next user's worklist; a desired user action for a radiologist may include a second image review of the acquired images comprising one or more of medical condition evaluation and diagnosis, and adding patient to a next user's worklist; and a desired user action of the clinician may include a third image review comprising diagnosis confirmation and treatment plan determination, and conveying diagnosis and treatment plan to the patient.

Next, at 504, the method 500 includes identifying a current step in the user's workflow via the workflow tracker. For example, the workflow tracker may determine if the current user is performing one of image acquisition and image review. As discussed previously, the current step for the current user may be determined via the workflow tracker. For example, image acquisition step may be identified based on one or more of user indication, launching an acquisition interface, and indication from the acquisition system; and image review may be determined based on one or more of user indication of review and monitoring a review and/or annotation field for the patient by the current user.

Next, the method 500 proceeds to 506. At 506, the method 500 includes identifying one or more possible future actions for the user based on the current workflow step. The identification of one or more future actions may be based on the desired user workflow and the current step in the user's workflow.

In one example, when the user is a technologist and the current step is an image acquisition phase, the method 500 may identify one or more future actions to include the first image review comprising one or more initial findings from the acquired images, and adding patient to a next user's worklist. If the technologist has completed image acquisition, and the current step is image review, the method 500 may identify one or more future actions to include completion of image review (e.g., indication of initial findings) and adding patient to the next user's worklist.

In another example, when the user is a radiologist who is not involved in acquiring images and the current step is the second image review, the method 500 may identify one or more future actions to include completion of the second image review (e.g., interpretation of acquired images, medical condition indication, diagnosis, etc.) and adding patient to the next user's worklist.

In yet another example, when the user is a clinician and the current step is third image review, the method 500 may identify one or more future actions to include completion of third review and conveying diagnosis and treatment plan to the patient.

Next, upon identifying one or more future actions based on current user and current workflow step, the method 500 proceeds to 508. At 508, the method 500 includes providing one or more recommendations to the user based on the identified one or more future actions. In one example, when the current user is the technologist acquiring images, upon determining that the image acquisition is complete, the method 500 may indicate to the user that one or more of image review and adding patient to the next user's worklist is pending. In another example, when the current user is the radiologist performing review/interpretation of acquired images, the method 500 may indicate to the user that adding patient to the worklist is pending. In yet another example, when the current user is the clinician/ordering physician, and the medical condition is confirmed, the method 500 may indicate to the user to convey diagnosis and treatment plan to the patient.

Further, in some examples, based on the typical workflow of radiologist and the completed steps during the current user (radiologist) workflow, (e.g., image selection, image review), predicted next steps may include adding the patient to the worklist of the clinician for diagnosis based on the generated report and/or adding the patient to the worklist of the technologist for further imaging.

In another example, based on the tracked workflow of the technologist, the workflow tracker system may identify that the technologist has completed imaging the left side of the patient's neck. The algorithm may determine (based on the saved worklist request of the ordering clinician) that the prescribed imaging protocol includes imaging the left side, the right side, and the front of the patient's neck. Thus, possible future actions may include imaging the right side of the patient's neck, imaging the front of the patient's neck, and/or storing the acquired images on the workflow tracker system.

In this way, based on current user and current workflow step of the user, one or more recommendations for next steps to be performed by the current user may be provided to the user to enable user to complete their workflow within the imaging workflow for the patient. Thus, the workflow tracker keeps track of a plurality of care provider workflow in the overall imaging workflow of the patient, and provides suggestions/recommendations to enable each of the plurality of care providers to complete their individual workflow while keeping track of the overall patient workflow as the patient imaging workflow moves from image acquisition to providing diagnosis and treatment plan to the patient.

It will be appreciated that the method 500 may be executed concurrently with method 400. Thus, while method 400 updates one or more image acquisition information, a patient information, and a review information as the current user performs their work flow, and provides indications when respective steps in the workflow of the patient (and thus, current user workflow) are completed, the method 500 tracks the current step and provides recommendations for the next step.

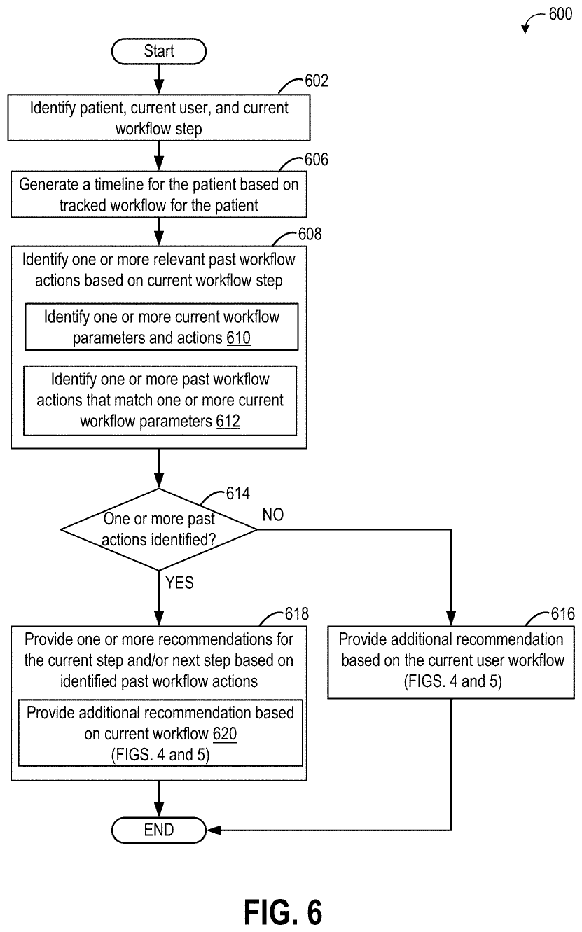

In addition to updating workflow information, providing completion indications at various steps, and providing recommendations for the next step, the workflow tracker application may evaluate one or more previous workflows (including previous imaging workflow, previous non-imaging workflow, etc.) for a patient, and provide recommendations to the user involved in the current imaging workflow of the patient based on the one or more previous workflows of the patient, as discussed below at FIG. 6.

Turning now to FIG. 6, it shows a high-level flowchart illustrating an exemplary method 600 for providing one or more recommendations to the current user involved in the current imaging workflow of the patient based on one or more previous tracked workflows of the patient. The method 600 may be implemented by a processing system, such as controller 148 at FIG. 1, an edge device connected to the processing system, a cloud in communication with the processing system, or any appropriate combination thereof. The method 600 is described with regard to the systems and components of FIGS. 1, 2, 3A, and 3B, although it should be appreciated that method 600 may be implemented with other systems and components without departing from the scope of the present disclosure. The method 600 may be executed in response to launching the workflow tracker application) on a workstation, such as workstation 214 at FIG. 2.

Method 600 begins at 602. At 602, the method 600 includes identifying a current patient, a current user, and a current workflow step in the workflow of the current user. The current patient and the current user may be identified as discussed at 402 at FIG. 4, and the current workflow step may be identified as discussed at 502 at FIG. 5, and thus, the above steps will not be repeated for brevity.

Next, the method 600 proceeds to 606. At 606, the method 600 includes generating a timeline for the current patient based on one or more previous tracked workflow for the patient and current workflow for the patient. The one or more previous tracked workflows for the patient may be obtained from a corresponding patient workflow tracker module for the patient, such as module 151 at FIG. 1, stored in the workflow tracker server system. The one or more previous tracked workflows for the patient may include imaging workflows and non-imaging workflows. For example, the one or more previous workflows may include workflows for the patient that was performed during previous clinical visits at different time periods. Thus, the one or more previous workflows may include one or more previous actions (e.g., previous image acquisition, reviews, diagnosis, and treatment plan) that were performed for the patient, and information (e.g., acquisition information and review information for each user (that is, healthcare provider) involved in the one or more previous workflows. Further, the one or more previous workflows may be performed by the same healthcare provider, different healthcare provider, or a combination thereof. Thus, the timeline may include all of the tracked workflows for the patient including one or more previous workflows and current workflows, and may further include one or more information related to each user involved in the one or more previous workflows. An example timeline that may be generated for the patient is shown at FIG. 7.

In this way, by implementing a workflow tracker one or more previous workflows for the patient may be accessed at any time as desired.

Next, upon generating the timeline, the method 600 proceeds to 608. At 608, the method 600 includes identifying one or more relevant past workflow actions and/or parameters from the one or more previous workflows that are relevant to current workflow. This includes, at 610, identifying one or more current workflow parameters and actions. The one or more current workflow parameters and actions may include one or more of image acquisition information, patient information, and review information stored and/or updated during the current workflow as discussed at FIG. 4. Thus, the one or more current workflow parameters and actions may include current user (that is, care provider performing the current workflow, which may be one of technologist, radiologist, clinician, depending on the current workflow step), nature of study (e.g., initial, follow-up, diagnostic, screening, etc.), imaging modality employed, body part imaged, etc.

Identifying one or more relevant past workflow actions further includes, at 612, identifying one or more past workflow actions and parameters that match and/or correlate with the identified one or more current workflow parameters. In one example, the identification one or more relevant past actions may be performed by an artificial intelligence based algorithm.

The artificial intelligence based algorithm may be implemented by a neural network that is comprised of an input layer that takes plurality of inputs (past workflow parameters and actions, and current workflow parameters and actions), one or more hidden layers that establish relationship between input and output layers, and an output layer that comprises one or more relevant past workflow actions that correlate with the current workflow parameters and actions. The network may be trained experimentally with a test data to compute the number of nodes, number of layers, and weights of each link. The trained network, which may be a deep neural network, is able to take in input features and compute relevant past workflow actions and parameters. The past work flow actions and parameters may be from a single previous workflow or from more than one previous workflow. An example output of relevant past workflow actions may include a previous workflow that was performed with respect to a previous body part of the patient. For example, the previous body part may be imaged with a previous imaging modality during the previous workflow of the patient, where the previous body part is the same as a current body part imaged during the current work flow and where the previous imaging modality is the same as the current imaging modality. In this way, the artificial intelligence algorithm may identify previous workflows and output relevant previous workflow and relevant past actions and parameters from the relevant previous workflow.

Next, method 600 proceeds to 614. At 614, the method 600 includes determining if one or more previous workflows are identified. If the answer is YES, method 600 proceeds to 618.

At 618, the method 600 includes providing one or more recommendations to the current user, via the workstation interface, for one or more of the current workflow step and future workflow step based on the past actions and parameters from the one or more previous workflows identified. Referring to the same body part and same imaging modality example at 612, a non-limiting example of recommendation may be a type of protocol to use during current imaging. For example, during the previous workflow, a preferred acquisition protocol may be established for the same body part of the patient with same imaging modality, and as a result, the recommendation may include an indication to the user regarding the established acquisition protocol. Based on the recommendation, the user may be able to obtain high quality images with high efficiency. As a result, patient care is improved.

In another example, the current workflow step of the user may include x-ray imaging the patient's left arm for a second time, with an initial diagnostic imaging also performed by the current user. The method 600 may output recommendations for how the x-ray imaging of the patient's left arm should be performed based on the patient worklist and stored data pertaining to the first time the patient's left arm was imaged (e.g., the protocol used, patient positioning, images reviewed, radiology report generated, reason for imaging the patient's left arm a second time, etc.). For example, the method 600 may output recommendations that the current user position the patient where anteroposterior and lateral images of the left forearm are obtained during diagnostic imaging based on the prescribed protocol issued by the collaborative user who ordered the second round of imaging. The current user may utilize the past information to improve imaging quality and efficiency. An exemplary use-case scenario is further illustrated below with respect to FIG. 7.

Further, providing one or more recommendations may include, at 620, providing additional recommendation based on current workflow as discussed at FIGS. 4 and 5.

Returning to 614, if one or more previous relevant workflows are not identified, the answer at 614 is NO, and the method 600 proceeds to 616 to provide additional recommendation based on current workflow as discussed at FIGS. 4 and 5.

The method 600 then ends.

FIG. 7 shows an exemplary timeline 700 for a patient that may be generated based on one or more tracked workflows for the patient. The one or more tracked workflows may include one or more previous workflows and one or more current workflows. The one or more tracked workflows may be generated for the patient, via a workflow tracker application, such as workflow tracker 275 shown at FIG. 3A, and a workflow tracker server system, such as workflow tracker server system 152 at FIG. 1. For example, during each visit to one or more care providers, a corresponding patient workflow may be tracked via the workflow tracker.

The timeline 700 may include a first patient workflow 710, a second patient workflow 720, a third patient workflow 730, and a fourth patient workflow 740. The first, second, and third patient workflows 710, 720, and 730 may be past patient workflows and the fourth patient workflow 740 may be a current patient workflow. The workflow tracker may be implemented to provide one or more recommendations for the current patient workflow 740 based on the past first, second, and third workflows.

The first patient workflow 710 may be a first imaging workflow, and may include a technologist workflow period u1, a radiologist workflow period u2 and a clinician workflow period u3. The technologist may acquire one or more MRI images of a head of the patient during the first patient workflow, the radiologist may interpret the one or more MRI images acquired by the technologist, and the clinician may confirm a diagnosis indicated by the technologist, and provide a treatment plan to the patient. Similarly, the second patient workflow 720 may include a technologist workflow period u4, a radiologist workflow period u5 and a clinician workflow period u6; the third patient workflow 730 may include a technologist workflow period u7, a radiologist workflow period u8 and a clinician workflow period u9; and the fourth patient workflow may include a technologist workflow period u10, a radiologist workflow period u11 and a clinician workflow period u12.

During the first patient workflow 710, a first head MRI may be performed utilizing a first acquisition protocol by a technologist during the technologist workflow period u1. However, upon review by a clinician during the clinician workflow period u3, the clinician may have ordered a second head MRI to be performed with a second different acquisition protocol to enable better diagnosis and treatment. Accordingly, the second head MRI may be performed and the second patient workflow 720 may be generated. During the second patient workflow 720, the second head MRI may be performed with the second acquisition protocol. The second head MRI with the second MRI may be indicated, by a clinician during the clinician review period u6, that the second acquisition protocol is the preferred protocol for the patient. Further, the clinician during the workflow period u6 may provide a treatment plan to the patient and prescribe a follow-up MRI after a later time point t1.

Prior to t1, the patient may be admitted for a leg x-ray, to evaluate a potential fracture, for example. Thus, the third workflow 730 may be generated for the patient corresponding to x-ray imaging of the leg for the patient.

After t1, the patient may be admitted for the follow-up MRI prescribed during the second workflow. Accordingly, the fourth workflow 740 for the patient may be initiated. During the technologist workflow period u10 of the fourth workflow 740, the workflow tracker may evaluate one or more past patient workflows 710, 720, and 730 to identify one or more relevant workflows with respect to the current workflow 740. The workflow tracker may identify one or more common parameters between the current workflow 740 and one or more past workflows 710, 720, and 730 to identify relevant workflow patterns. As a result, workflows 710 and 720 may be identified as relevant workflows as they correspond to the same imaging modality and the same body part as the current work flow 740, and based on the follow-up indication by the clinician during u6. Further, the workflow tracker may identify that the second different protocol for MRI has been indicated as the preferred protocol for the patient, and may provide a recommendation at 742 during u10 to the technologist. The recommendation may include an indication of the preferred protocol that may be used during the current workflow. In this way, based on one or more past actions and parameters, recommendations may be provided during the current workflow to enable more efficient workflow and increase confidence in imaging and diagnosis.

If past workflows were not tracked or considered, during the current workflow, the information as to the preferred protocol may be lost, and may require unnecessary recalls and repetitions before the desired imaging is performed.

While FIGS. 6 and 7 illustrate tracking overall patient workflow based on patient workflow timeline that includes specific user workflows, in some examples, timeline for a user may be generated, and recommendations may be provided based on past action, such as collaborations, for the user.

In some examples, based on the generated user timeline, the typical workflow of the user, and the generated timelines of other collaborative care providers, the workflow tracker system may output a recommendation for the most time effective next step with the respect to the patient workflow. In one example, the current user may be in the process of completing diagnostic imaging of the patient. Other collaborative care providers of the patient may include a radiologist, a clinician, a pathologist, and a technician. The patient worklist may still include uncompleted tasks of diagnostic image review, electrocardiogram (ECG) testing, blood testing, and patient diagnosis/treatment. The real-time tracked workflow and generated timeline of the radiologist may indicate that review of the acquired images will begin forty-five minutes after they have been acquired, as the radiologist has two other workflow tasks to complete before beginning the review. The real-time tracked workflow and generated timeline of the pathologist may indicate the pathologist will be available for blood testing in fifteen minutes. The real-time tracked workflow and generated timeline of the technician and the clinician may show that both are available for the next thirty minutes. Thus, the workflow tracker system may output a recommendation to the current user to transport/take the patient to an exam room where the technician may perform an ECG testing as the pathologist and radiologist are currently performing other tasks and the clinician, while free, cannot make a diagnosis without the radiology report and completed patient testing.

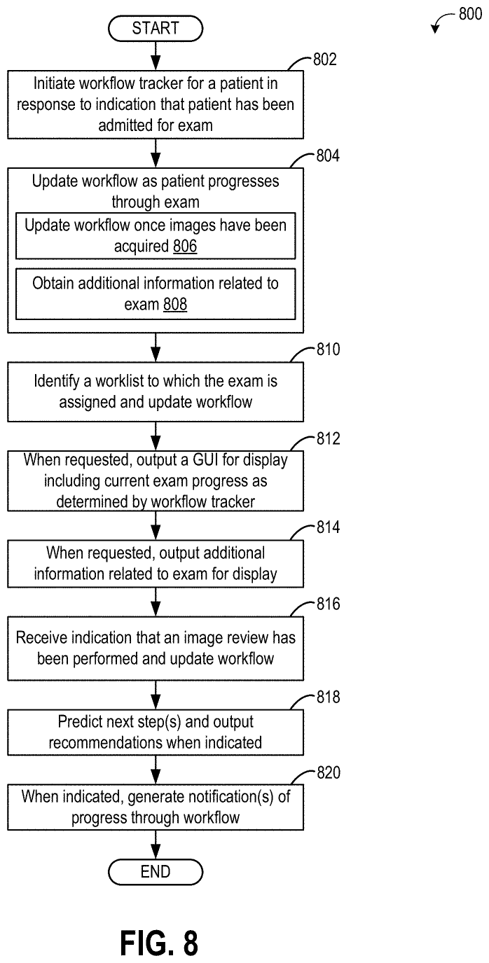

FIG. 8 is a high-level flowchart of an exemplary method 800 for tracking the workflow of a specific patient case in real-time, according to an embodiment of the present disclosure. Method 800 is described with respect to the system and components described above with respect to FIGS. 1 and 2 but could also be carried out by and/or with other systems/components without departing from the scope of this disclosure. The method 800 may be implemented by a processing system, such as controller 148 at FIG. 1, an edge device connected to the processing system, a cloud in communication with the processing system, or any appropriate combination thereof. The method 800 is described with regard to the systems and components of FIGS. 1, 2, 3A, and 3B, although it should be appreciated that method 600 may be implemented with other systems and components without departing from the scope of the present disclosure. The method 600 may be executed in response to launching the workflow tracker application) on a workstation, such as workstation 214 at FIG. 2. Method 800 may include tracking a patient workflow in real-time, and outputting a continually updated timeline of the workflow as a workflow user interface.

At 802, a workflow tracker for a patient may be initiated in response to an indication that the patient has been admitted for an exam. In some embodiments, the indication may be a signal sent from a camera, an audio sensor, a self-serve check-in station, and/or a user interface (user interface 226 of FIG. 2) communicatively coupled to the workflow tracker system. For example, the camera may track the time of patient entrance into the facility and the audio sensor may be used to determine who the patient is using natural language processing (NLP). In another example, a signal may be sent from the user interface used by staff during the patient admittance process. Once a signal has been sent to the workflow tracker system, the system may generate a workflow tracker for the patient.

At 804, the workflow of the patient may be updated as the patient progresses through the exam. From the time of admittance when the workflow tracker is initiated, the workflow tracker system may generate and track a patient worklist in real-time. For example, the patient may be tracked via signals sent to the workflow tracker system as the patient moves throughout the facility, interacts with care providers, and undergoes various exams and/or tests. The worklist and workflow may be continually updated via the signals sent to the system. Updating the patient workflow may include, at 806, updating the workflow once images have been acquired. For example, after the completion of an imaging exam, images or a signal indicating that image acquisition has been completed may be automatically sent from an imaging system (e.g., an MRI system, a CT system), a PACS (e.g., PACS 208 of FIG. 2), a data center (e.g., data center 212 of FIG. 2), and/or a network (e.g., network 154 of FIG. 1) to the workflow tracker system. In some examples, the completion of image acquisition may be manually entered into the workflow tracker system by a user via a user interface. Updating the patient workflow may further include, at 808, obtaining additional information related to the exam. Additional information may include when/where the exam was completed, who was the ordering physician, who performed the exam, the parameters/protocol employed during the exam, contact information for staff involved in the ordering/completion of the exam, the number of images taken, why the exam was performed, if the exam was previously performed, and so on. The additional information may be automatically and/or manually input into the workflow tracker. For example, information data may be automatically output from the data center, a radiology information system (e.g., radiology information system 206 of FIG. 2), and/or another information system communicatively coupled to the workflow tracker system.

At 810, a worklist to which the exam is assigned may be identified and the workflow updated. The worklist may be the worklist for the patient case and/or a worklist generated for a care provider that includes tasks for multiple patients. After the exam has been completed, the worklist may be updated indicating the task or task associated with the exam as being completed. For example, the patient worklist may include tasks of a MRI exam, a blood test, and diagnosis review with a clinician. The patient worklist may be updated to indicate what tasks are still left to be performed with regard to the patient workflow. Thus, after completion of the MRI exam, the worklist may be updated to indicate that the patient still requires blood testing and that the patient has not received a diagnosis. Alternatively, after completion of the MRI exam, the worklist of a technician who performed the exam may be updated to indicate that task as completed for that particular patient.

At 812, when requested, a graphical user interface (GUI) may be output for display that includes current exam progress as determined by the workflow tracker. The GUI may be workflow tracker interface as shown and described with respect to FIGS. 3A and 3B. The GUI may include different sections that correlate to stages within the patient workflow following admittance. For example, if the patient worklist includes an imaging exam, the GUI may include sections pertaining to the timeline of steps involved in the imaging exam. In one example, the GUI may include an images taken section, an added to worklist section, an image review section, and a generate report section. The current step of the imaging process may then be indicated to the user by changing a background color of the section of the GUI that correlates to the current step. For example, if the acquired patient images are currently undergoing review, the background of the image review section may be filled with grey and the background of all other sections of the GUI may be white. Thus, the user may get a clear visual and understand at a glance where the patient is in the current workflow. In some examples, the section correlating to the current workflow step may be otherwise suitably indicated within the GUI. For example, the section of the current workflow step may be larger than the other sections of the GUI. In another example, the shape of the section of the current workflow step may be different than the other sections of the GUI (e.g., the section of the current workflow step may be diamond-shaped while the other sections of the GUI may be rectangular. In some examples, completed steps, the current step, and future steps of the patient workflow may be distinguished from each other (e.g., completed step sections may be a different shape/size color than that of the current step section, and the shape/size color of the future step sections may be different than that of the current step and completed step sections).

At 814, when requested, additional information related to the exam may be output for display. The additional information may include details of the current workflow step, future workflow steps, and/or completed workflow steps. For example, in the example described above, after patient images have been acquired, the user may be able to select or hover over the images acquired section to obtain additional details pertaining to the acquired images. An example display of additional details pertaining to the acquired images are shown and described at FIGS. 11 and 12. Selection of or hovering over the images acquired section may trigger a window to be displayed that includes the name and contact information of the technician who performed the exam, the name and contact information of the physician who requested the exam, and the number of images acquired. In another example, once the generate report step has been completed, the user may select the generate report section of the GUI to view the report, who generated the report, when the report was generated, and so on.

At 816, an indication that an image review has been performed may be received and the workflow updated. As previously described, the workflow tracker system may automatically receive a signal output from a system involved in the image review indicating that the review has been complete. In some examples, the individual who performed the image review may manually enter the task as complete thereby updating the workflow.

At 818, method 800 may predict next step(s) and output recommendations when indicated. For example, in response to a request from the user and/or automatically, an algorithm may be employed to determine and output possible next steps based on the completed steps of the patient workflow, the medical history of the patient, the typical workflow for similar patients, the workflows of collaborative care providers of the patient, generated reports, and/or other data. For example, a patient workflow tracker may indicate that the patient has undergone an imaging exam, the images were reviewed but that a report has not been generated and the patient still requires blood testing. Thus, the algorithm may determine based on a current workflow of the radiologist who will generate the report, a current workflow of the pathologist who performed the blood testing, and the remaining tasks on the patient worklist that potential next steps may be report generation, blood testing, and/or multidisciplinary team review. The algorithm may output these predicted next steps to the user.

In some examples, the algorithm may indicate which may be the most time efficient predicted next step. For example, if the current workflow of the radiologist indicates that the radiologist may not be able to generate the report for one hour (e.g., as the radiologist is busy with other patients), the algorithm may output the performing blood testing with the pathologist as a preferred next step.

In another example, the algorithm may predict future next steps based on the completed patient worklist, the information generated from the completion of the patient worklist, the medical history of the patient, and/or the typical workflow for similar patients. For example, the patient may have received a leg x-ray where it was determined the patient had a broken femur and received a cast. The algorithm may then output next steps that include scheduling follow-up appointments for imaging, cast removal, physical therapy, etc. If the algorithm determines the patient has broken the same femur multiple times (e.g., based on the patient medical history), the algorithm may output recommendations for osteoporosis or hone density testing. In some examples, the recommendations may be listed as optional next steps within the patient worklist on a collaborative interface that the user may accept or reject via direct input (e.g., via a keyboard, a voice command module, or touchscreen communicatively coupled to a display device).

At 820, when indicated, method 800 may generate notification(s) of progress through the workflow. For example, as the workflow tracker is updated in real-time, a notification may be output to collaborative care providers of the patient whenever a step of the patient worklist is completed. The notifications may be output to a care provider device (e.g., care provider device 134 of FIG. 1) as visual or audio alerts. In some examples, the generated notifications may indicate what tasks still remain on the patient worklist. For example, a notification may be issued only to the care providers who are performing the remaining worklist tasks.

The method 800 then ends.

FIG. 9 shows a block diagram illustrating exemplary user workflows for a patient tracked by a workflow tracker application, such as workflow tracker 275 at FIG. 2. In particular, a patient imaging workflow 900 including a technologist workflow 902 and a radiologist workflow 904 are illustrated. Although not shown herein, it will be appreciated that the patient imaging workflow may include one or more additional workflow for one or more additional collaborative/health care providers. As an example, during diagnostic imaging, a technologist may acquire patient images, a radiologist may review the acquired images and generate a radiology report, and the clinician may review the generated radiology report to determine a patient diagnosis. The block diagram illustrates exemplary tracking of the technologist workflow and radiologist workflow with respect to imaging workflow for a patient (patient 1). The technologist may be operating in an acquisition workstation via an acquisition interface of an imaging modality. The workflow tracker application may be launched with the acquisition interface, and thus tracking of the technologist workflow for the patient is enabled via the workflow tracker. The radiologist may be operating in a different clinical workstation (e.g., PACS workstation) via a clinical viewer interface, for example. The workflow tracker application may also be implemented and launched with the viewer interface, and thus tracking of the radiologist workflow with respect to the patient is enabled via the workflow tracker. Similarly, tracking of one or more additional collaborative/healthcare providers may be enabled via the workflow tracker. Thus, the workflow tracker tracks a plurality of workflows for the patient. While the examples herein are illustrated for a single patient, the workflow tracker may track workflows for a plurality of patients visiting a healthcare system, and also among different healthcare systems thereby enabling seamless transfer of information between different care providers for the patient.

The workflow tracker may track the technologist workflow 902 over time for a first image (e.g., Image 1), a second image (e.g., Image 2), and so on up to an nth image (e.g., Image N) are completed in real-time. For example, the tracked technologist workflow 902 shows that the steps of image acquisition, adding the acquired image to the workflow tracker, and adding the patient to the worklist have all been completed for Image 1, as indicated by a work progress bar 908 in relation to a general technologist workflow timeline 910. The last completed workflow step is indicated by an upside triangle 912 on the work progress bar 908, with the position of the upside down triangle 912 aligned to the corresponding step on the technologist workflow timeline 910 (e.g., patient added to worklist). In another example, the technologist (user 1) has acquired and added Image 2 to the workflow tracker as indicated by a work progress bar 914 for Image 2, whereas Image N has been acquired but is not stored and/or updated by the workflow tracker.

As the workflow timeline 910 of the tracked technologist workflow 902 for each image is completed, a next current user (user 2) may be notified of next steps as the patient is added to the worklist for that user. For example, the patient may be added to a worklist of the radiologist (user 2) for review of Image 1. The radiologist workflow 904 may include a general radiologist workflow timeline 910 that includes steps of patient selection, image selection, image review/report generation, and adding the patient to the worklist. As shown by a work progress bar 918 for Image 1, the radiologist has completed all steps and the patient has been added to the worklist of the next current user. Alternatively, only patient selection has occurred for Image 2 as the technologist has not yet completed the step of adding the image to tracker where it may be viewed by the radiologist. The patient workflow may further include a clinician workflow (not shown) that is also tracked by the workflow tracker.

FIG. 10 is a high-level flow chart illustrating an exemplary method 1000 for facilitating communication amongst and outputting notifications to collaborative users and peers who have been part of a patient's case based on the tracked workflow of current users. The method 1000 may be implemented by a processing system, such as controller 148 at FIG. 1, an edge device connected to the processing system, a cloud in communication with the processing system, or any appropriate combination thereof. The method 1000 is described with regard to the systems and components of FIGS. 1 and 2, although it should be appreciated that method 1000 may be implemented with other systems and components without departing from the scope of the present disclosure. The method 1000 may be executed in response to launching the workflow tracker application) on a workstation, such as workstation 214 at FIG. 2.

Method 1000 begins at 1002. At 1002, method 1000 includes receiving a notification that a patient has been admitted to the medical facility. The notification may be received from the healthcare information infrastructure and may include a patient identifier, patient state (e.g., the condition for which the patient is being admitted), and care provider information. The care provider information may include identifiers of various care providers (such as clinicians, technologists, and radiologists) that are currently attending to the patient as well as information for care providers the patient has seen in the past.

At 1004, method 1000 includes generating a communication channel and a workflow tracker dashboard for the patient. In order to generate the communication channel, verified care providers of the patient (e.g., as indicated by the notification from the healthcare information infrastructure) may be joined to the communication channel, as indicated at 1006. The communication channel may facilitate text and/or rich-media based messages to be sent among all the verified care providers that are joined to the communication channel. Generating the workflow tracker dashboard may include configuring the dashboard based on the workflow of all current users (e.g., care providers currently administering to the patient) in real-time and generating a continually updated patient worklist as well as adding contact information for and results from other verified care providers to the dashboard, as indicated at 1008.

The workflow tracker dashboard may be a graphical user interface that facilitates display of patient medical information, as further shown and described with respect to FIGS. 11-13, including: medical history; acquired patient images; detailed acquisition information related to acquired patient images (e.g., where and when the images were acquired, the protocol used in acquisition, who acquired the images, the ordering provider, etc.); contact information for all verified care providers; location information for all current users; completed steps of a current user's workflow with regard to the patient (e.g., images reviewed, patient added to the worklist, etc.); and a patient worklist updated in real-time based on the workflow of current users that may include next steps. The dashboard may also include relevant/desired messages from the communication thread. Verified care providers joined to the communication channel may view the workflow tracker dashboard as it is updated in real-time with workflow information from/for each of the current users.

Further, generating the communication channel may include connecting other applications to the communication channel, as indicated at 1010. For example, the workflow tracker server may connect to a global positioning system (GPS) application that may be stored on mobile devices (e.g., a smart phone, a smart watch, a tablet) of collaborative users. By connecting the GPS application to the tracker server, the physical location of all current users may be tracked (within and outside of the medical facility) in real-time and displayed on the workflow tracker dashboard. In some examples, the workflow tracker server may connect to a map-based application. The map-based application may provide navigational resources for getting to the patient's current location from another medical facility and/or directions for transporting the patient to another facility based on the current user workflow and next steps within the patient worklist. In some examples, the workflow tracker server may connect to a chat or voice call application that allows previous care providers/current users to communicate (e.g., via text or voice messages, voice calls) one-on-one with other verified care providers of the patient. Thus, the patient's case may be discussed in real-time with relation to the workflow of the current user(s). Further, the workflow tracker server may connect with a mobile application that allows verified care providers to send/receive notifications related to the patient and/or changes within the current user's workflow on a mobile device in real-time. The location information, connection to chat and/or voice call application, notification enablement, and other application enabled functions as discussed above may be provided via the workflow tracker dashboard.

At 1012, method 1000 includes receiving text- and/or rich-media-based messages from the participants on the communication channel, including previous patient care providers and current users. During the course of patient care (e.g., the workflow of all current users), care providers may communicate with each other on the communication channel via messages of the communication thread to coordinate care, give care instructions, and/or confirm appropriate care is being carried out. Further, verified care providers may send requests to the via the communication thread for various information related to the patient care, including patient medical history, care guidelines, predicted future patient state, recommended lab tests, etc. Further still, care providers may send notifications via the communication thread of changes in patient state, patient medical history, patient care guidelines, predicted future patient states, lab test status, etc.

The messages sent from a care provider may be sent from a care provider device (e.g., device 134) and received at the server system via a suitable connection (e.g., wired or wireless, such as via the Internet). In some examples, the messages may be automatically generated by a virtual healthcare assistant (VHA) using a data mining algorithm as the patient's worklist is continually updated in real-time. The messages sent may be stored and executed on the server system, a cloud, and/or a remote device. As used herein, messages may refer to any suitable information sent and received on the communication thread, including but not limited to text messages (entered via typing, touch, or stylus input, voice input, or automatically generated by a virtual healthcare assistant), images, voice messages (e.g., recordings of voice input), and videos.

At 1014, method 1000 includes distributing the received messages to other participants on the communication channel and saving the received messages as a communication thread. Each message that is sent to the server system may be tagged with various identifiers that identify the sender as well as the patient communication thread to which the message pertains (e.g., the patient identifier). The server system may then send the message to other participants of the communication channel, e.g., the care providers and/or VHAs that did not send the original message, and save the message as part of a saved communication thread. The saved communication thread may then be viewed by other users at other times, retrieved in response to a user request to view some or all of the communication thread, etc. However, in some examples, the device from which the original message was sent (e.g., the care provider device) may send the message to all other participants on the communication channel, and thus the server system may not distribute the message to the other participants.

At 1016, method 1000 includes outputting the communication thread and/or dashboard for display when prompted. In an example, the prompt may include an explicit request to view the communication thread and/or workflow tracker dashboard for the patient, entered by selection of an appropriate link/control button on the workflow tracker dashboard or selection of the patient's communication thread from a collaborative interface, as indicated at 1018. For example, a message button may be displayed via the collaborative interface, and selection of the message button may trigger display of the communication thread and/or workflow tracker dashboard for that patient. In another example, a patient link may be selected to launch the communication thread or dashboard from a collaborative system interface. In an example, the communication thread and/or workflow tracker dashboard may be output for display automatically in response stored instructions within the workflow tracker system, as indicated at 1020. For example, the workflow tracker system may automatically output the communication thread every time the thread receives a new message. In some examples, the workflow tracker system may automatically output the workflow tracker dashboard after patient admittance to all current users.

At 1022, method 1000 includes outputting notifications to relevant care providers with current user(s) workflow changes in real-time. Relevant care providers may include all current users as well as any previous care providers that may elect to receive notifications of workflow changes. For example, when an item on the patient worklist has been completed, a notification may be issued to all relevant care providers. In another example, when a new item has been added to the patient worklist, a notification may be issued to all relevant care providers. In another example, a notification may be issued when the patient is moved (e.g., to another medical facility, to an exam room) or discharged. Method 1000 then returns.

Turning to FIG. 11, a viewer interface 1100 that includes the workflow tracker interface 275 is illustrated in FIG. 11. Upon selection of the Image Review control button 314, an Image Viewer page 1102 may open within the collaborative system interface 1100. The Image Viewer page 1102 may include a banner 1104 that spans the top of the page listing basic patient information (e.g., the patient name, date of birth (DOB), medical record number (MRN), age, gender) and a selectable list 1106 of all the acquired images for the patient. The selectable list 1106 may include images acquired from the current visit of the patient as well all images acquired from previous care providers. The selectable list 1106 may include the date the images were acquired, the acquisition modality (e.g., a CT scan, MRI or x-ray imaging), and what body part was imaged (e.g., pelvis, chest, abdomen). Upon selection of a desired set of images from the selectable list 1106 (e.g., a CT scan of the patient's abdomen/chest/pelvis taken on Jan. 7, 2019), the acquired images may be displayed within the Image Viewer page 1102 for user review. Additional image acquisition information may be displayed for any image set by selecting the Images Taken control button 310. Upon selection of the Images Taken control button 310, an information window 1108 may be launched within the collaborative system interface 1100. An enlarged view 1200 of the information window 1108 is shown in FIG. 12.

The information window 1108 may display location information 1202 that includes the name of medical facility (e.g., Northwestern Hospital) where the images were acquired. In some examples, the displayed name of the medical facility may be a selectable to launch a map that includes the location and address of the medical facility. For example, the selectable name of the medical facility may be linked to a navigation or map-based application that may provide the user with directions from the current facility to the medical facility where the acquired images were obtained. The information window 1108 may further include a date 1204 the images were acquired.

In some examples, the date may be selectable to launch a calendar-based application that displays all of the image acquisition dates of the patient within a calendar system. For example, upon selection of the date 1204, a calendar showing all the months for the current year may be displayed. The dates when patient images were acquired may be marked within the calendar, with the date pertaining to the currently displayed images highlighted. In some examples, the acquisition dates within the calendar may selectable and automatically launch/open the set of images acquired on the selected date within the Image Viewer page 1102 or the collaborative system interface 1100.

The information window 1108 may include images taken information 1206 where the number of images acquired on the date 1304 may be listed (e.g., Images Taken: 1100 may indicate that 1100 images were acquired on the date 1204). The information window 1108 may include protocol detail information 1208 that may be selectable (e.g., a link, an icon, a control button) to launch an information window that includes detailed information related to the protocol used during image acquisition. For example, protocol detail information 1208 may include the name of any standard protocol employed (e.g., protocol 24, protocol 36), the modality used, dose applied, number of scans, scan length, body part targeted, gantry rotation speed, and so on.

Further, the information window 1108 may include care provider information 1210 for all of the care providers that assisted with patient care on the date 1204 the images were acquired. Care provider information 1204 may include a list of all care providers' names (e.g., Dr. Amorim, Dr. H. Bharwarl, Dr. Keizer). In some examples, the care provider names may be selectable to launch display of additional information for the selected care provider. For example, selection of a care provider (e.g., Dr. Keizer) may launch the display of a webpage listing the care provider's specialties, background, educational history, and so on (e.g., selection of the care provider may launch the display of the care provider's information page hosted on the website of the medical facility where the care provider is employed). In some examples, selection of the care provider may launch a display that includes all actions and/or reports generated by the care provider for the patient on the date 1204 the images were acquired (e.g., a workflow or worklist for the care provider for the date 1204 may be displayed). Additionally, care provider information 1210 may include thumbnail images 1212 that display a picture of each care provider adjacent to and aligned with the care provider's name. For example, a thumbnail image 1222 may display a picture of Dr. Keizer.

Further, adjacent to each care provider's name listed within the care provider information 1210 may be selectable telephone icons 1214 and selectable chat icons 1216. For example, the two icons may appear linearly adjacent to each care provider's name. Selection of one of the telephone icons 1214 may launch an application where the current user may call and/or leave a voice message for the care provider whose name appears adjacent to the selected telephone icon. For example, selection of a telephone icon 1218 may call a mobile device (e.g., a tablet, a mobile phone, a smart watch) or landline associated with Dr. Keizer. Selection of one of the chat icons 1216 may launch an application where the current user may send text- and/or media-rich messages to the care provider whose name appears adjacent to the selected chat icon. For example, selection of a chat icon 1220 may launch a chat-based application on a display device (e.g., a smart phone, a smart watch, a tablet) associated with Dr. Keizer.

FIG. 13 shows a viewer interface 1300 that includes the workflow tracker interface 275. Upon selection of the Added to Worklist control button 312, a Worklist page 1302 may be launched within the viewer interface 1300. The Worklist page 1302 may include a worklist 1304. The worklist 1304 shown is listing of all patients who has done imaging and review is required by radiologist or other specialty.

The workflow tracker interface 275 may indicate workflow progress for a current patient selected from the worklist 1304. If the current patient selection is changed to a second patient, the workflow tracker interface 275 may be updated to display the workflow progress of the second patient.

Further, an upper portion of the worklist interface including a priority indication (percentage wheel), a modality pie chart, and indications of body parts imaged are filters to narrow the worklist.

The worklist 1304 may include a series of rows and labelled columns. Each row may correspond to a step within the workflow of a current user with respect to a selected patient's care, with the worklist 1304 comprised of the workflow steps for all collaborative users for the selected patient. Each labelled column may correspond to information related to the workflow step listed within the worklist 1304. For example, the series of labelled columns may include a Stat column 1306, a Time/Date column 1308, a Modality column 1310, a Study Description column 1312, a Patient Name column 1314, a Reason for Exam column 1316, and an Ordering Physician column 1318. Information within the labelled columns for each workflow step within the worklist 1304 may be automatically input (e.g., via an algorithm) and/or directly input by the user (e.g., via a keyboard, a voice command module, or touchscreen communicatively coupled to a display device).

The Stat column 1306 may indicate critical cases which appeared in the worklist For example, an exclamation mark 1320 may be used to indicate if the workflow step is of high priority (e.g., the patient's health may deteriorate if the workflow step is not completed within a relatively short time span, the patient is leaving soon and cannot come to another appointment for several months, etc.). Thus, if the workflow step within the worklist 1304 does not have an exclamation mark in the Stat column 1306, the workflow step may be considered to be of lower priority than workflow steps that have an exclamation mark in the Stat column 1306. In some examples, the priority of the workflow step within the worklist 1304 may be indicated by other suitable means (e.g., the Stat column 1306 may include labels of high, medium, and low indicating if the workflow step is of high, medium, or low priority, respectively). In some examples, The Stat column 1306 may include a selectable arrow 1326 or icon that may re-order the worklist 1304 based on priority after selection (e.g., all workflow steps indicated as high priority may be located at the top of the worklist 1304).

Further, the status of the workflow step may be indicated by a closed lock icon 1322 or an open lock icon 1324 within the Stat column 1306. All rows of workflow steps within the worklist 1304 that have not occurred may have the closed lock icon 1322 within the Stat column 1306 indicating that the step has not yet been completed. Once a workflow step is completed, the closed lock icon 1322 within the Stat column 1306 may be changed to the open lock icon 1324 (e.g., via direct input and/or automatically) thereby indicating the workflow step has occurred/been finished. In some examples, one or multiple completed workflow steps (e.g., as indicated by the open lock icon 1324), may be selected (e.g., selectably highlighted or chosen amongst the other rows of the worklist 1304) by the user.

After the user has selected desired completed workflow steps, the Generate Report control button 316 may be selected to generate a report for each of the selected workflow steps. The generated report may be a summary of the completed workflow step that may include images, results, findings, protocols used, actions taken, collaborative user notes, care recommendations, and so on. In some examples, the generated report may be customized based on user preferences (e.g., the user may select from a drop-down menu what aspects of the completed workflow step are included in the generated report). In some examples, the status of the workflow step may be otherwise suitably indicated (e.g., the Stat column 1306 may include a designation of “complete” for completed steps, the background of rows of completed steps may appear green and/or the background of rows of steps that have not been completed may appear red).

The Time/Date column 1308 may include the time and date each workflow step is added to the worklist 1304. In some examples, the Time/Date column 1408 may be subdivided to include the time/date the workflow step was added to the worklist 1304 (e.g., 2:26 PM on Mar. 3, 2019) and the time/date the workflow step was completed (e.g., 4:09 PM on Mar. 3, 2019). The Modality column 1310 may indicate the diagnostic modality to be employed or that was employed for a given workflow step. For example, the Modality column 1310 may include a boxed icon 1328 that includes a two letter abbreviation for each modality. In one example, the two letter abbreviation “DX” may indicate that the workflow step includes diagnostic imaging. In another example, the two letter abbreviation “BT” may indicate that the workflow step includes diagnostic blood tests. In some examples, the modality may be otherwise suitably listed within the Modality column 1310 (e.g., the modalities may be indicated by representative picture icons within each row).

The Study Description column 1312 may include a brief description of each workflow step. For example, if the workflow step includes the patient undergoing a CT scan of the abdomen and pelvis, the Study Description column 1312 may include a description that states “CT Abdomen and Pelvis” for that workflow step. In some examples, the Study Description column 1312 may include more or less information about the workflow step. For example, in the above example, the positioning requested/used for the CT scan may be additionally included within the Study Description column 1312 so that the description may state “CT Abdomen and Pelvis—Supine.” The Patient Name column 1314 may list the first and last name of the patient for whom the worklist 1304 has been generated. The Reason for Exam column 1316 may list a brief description as to why the workflow step has been added to the worklist 1304. For example, the workflow step for the patient may include the description of “Xray Spine” within the Study Description column 1312 and the Reason for Exam column 1316 may include a description stating “Patient complaining of back pain after falling down a flight of stairs.” The Ordering Physician column 1318 may list the name of the care provider who requested the workflow step (e.g., Dr. Keizer). In some examples, the worklist 1304 may include more, less, and/or a different series of labelled columns. In one example, the worklist 1304 may not include the Modality column 1410. In a second example, the worklist may include a labelled column that indicates which care provider completed the workflow step.

Further, the Worklist page 1302 may include a list of collaborative users 1330. The list of collaborative users 1330 may include a selectable name of each current user and the user's occupation within the medical facility (e.g., Jeff McGee Radiologist, Glen Ford Clinician). Upon selection of a current user's name within the list of collaborative users 1330, a phone number 1332 of the selected current may be displayed as well as a selectable video icon 1334, a selectable phone icon 1336, and a selectable chat icon 1338. The video icon 1334, phone icon 1336, and chat icon 1438 may be selected to launch an application that may allow the user to video chat with the selected user, voice call and/or leave a voice message for the selected user, or chat with the selected user via text- and/or media-rich messages, respectively.

FIGS. 3A, 3B, and 11-13 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
receiving an indication that a patient has been admitted for an exam, and in response, initiating a workflow tracker for the patient;
updating the workflow tracker in real-time as the patient progresses through the exam; and
outputting a graphical user interface (GUI) for display on a display device, the GUI including an indication of current exam progress as determined by the workflow tracker.

2. The method of claim 1, wherein updating the workflow tracker in real-time as the patient progresses through the exam comprises updating the workflow tracker to indicate acquisition of one or more diagnostic images is complete.

3. The method of claim 2, wherein updating the workflow tracker in real-time as the patient progresses through the exam comprises updating the workflow tracker to indicate review of the one or more diagnostic images is to be performed.

4. The method of claim 3, wherein updating the workflow tracker in real-time as the patient progresses through the exam comprises updating the workflow tracker to indicate the review of the one or more diagnostic images is complete.

5. The method of claim 4, further comprising outputting a notification for display on the display device including a recommended next action, the notification output during review of the one or more diagnostic images and/or upon completion of the review of the one or more diagnostic images.

6. The method of claim 5, wherein the recommended next action includes a recommendation to generate a report corresponding to the review of the one or more diagnostic images and/or a recommendation to schedule a multi-disciplinary team review corresponding to the exam.

7. The method of claim 5, further comprising determining the recommended next action based on output from a recommendation model.

8. The method of claim 2, further comprising, responsive to a user input, outputting an information window for display on the display device, the information window including additional information related to the exam.

9. The method of claim 8, wherein the additional information comprises one or more of:
a number of diagnostic images acquired, a location where the one or more diagnostic images were acquired, a date of when the one or more diagnostic images were acquired, a protocol according to which the one or more diagnostic images were acquired, and care provider information.

10. The method of claim 1, further comprising outputting one or more notifications for one or more care providers, the one or more notifications indicating a certain phase of the exam has been completed and/or indicating a certain phase of the exam is to be performed next.

11. A method, comprising:
in response to a user initiating a medical exam for a patient, initiating a workflow tracker for the user;
monitoring a current step in a user workflow via the workflow tracker;
determining a next step in the user workflow via the workflow tracker; and
indicating, via a workflow tracker interface of a display device, the determined next step.

12. The method of claim 11, wherein the next step is determined based on a tracked workflow of the patient prior to initiating the image acquisition.

13. The method of claim 11, further comprising: in response to the user completing each step in the user workflow, outputting one or more notifications for one or more care providers, the one or more notifications indicating a corresponding step of the exam has been completed and/or indicating a subsequent step of the exam is to be performed next.

14. The method of claim 11, wherein the workflow tracker interface includes a plurality of control buttons, each of the plurality of control buttons corresponding to a workflow step in the user workflow.

15. The method of claim 14, further comprising: visually indicating, via the workflow tracker interface, a current status of one or more steps in the user workflow.

16. The method of claim 15, further comprising, responsive to a user input, outputting an information window for display on the display device, the information window including additional information related to one or more of the patient, the exam, and a tracked workflow of the patient, wherein the tracked workflow of the patient includes the user workflow.

17. The method of claim 16, wherein the additional information comprises one or more of:
a number of diagnostic images acquired, a location where the one or more diagnostic images were acquired, a date of when the one or more diagnostic images were acquired, a protocol according to which the one or more diagnostic images were acquired, and care provider information.

18. A system, comprising:
a display; and
a computing device operably coupled to the display and storing instructions executable to:
receiving an indication that a patient has been admitted for medical imaging, and in response, initiating a workflow tracker to track a medical imaging workflow for the patient;
updating the workflow tracker in real-time as the patient progresses through the medical imaging workflow; and
outputting a graphical user interface (GUI) for display on the display device, the GUI including an indication of the imaging workflow progress as determined by the workflow tracker;
wherein the GUI includes a plurality of control buttons corresponding to each step in the medical imaging workflow.

19. The system of claim 18, wherein the instructions are executable to, output an indication to a user performing the medical imaging via the display, the indication including a recommendation for one or more of a current step and a next step in the medical imaging workflow based on one or more previous tracked workflows of the patient.

20. The system of claim 19, wherein the instructions are executable to, responsive to a user request, outputting an information window for display on the display device, the information window including additional information related to the medical imaging workflow.
